(19) United States
(12) Patent Application Publication
(10) Pub. No.: US 2018/0303406 A1
(43) Pub. Date: Oct. 25, 2018

(54) SYSTEMS AND METHODS FOR CHARACTERIZING SKIN TYPE FOR AESTHETIC AND DERMATOLOGICAL TREATMENTS

(71) Applicant: Dominion Aesthetic Technologies, Inc., San Antonio, TX (US)

(72) Inventors: Robert E. McKinney, Winter Park, FL (US); Donald G. Herzog, Collingswood, NJ (US)

(21) Appl. No.: 15/962,443

(22) Filed: Apr. 25, 2018

Related U.S. Application Data
(60) Provisional application No. 62/602,463, filed on Apr. 25, 2017.

(51) Int. Cl. A61B 5/00; G06K 9/46

(52) U.S. Cl. CPC A61B 5/444; A61B 5/0082; A61B 2562/0242; A61B 5/72; G01N 2800/207; G06K 9/4652

(57) ABSTRACT

Systems for characterizing skin type including an illumination source configured to generate and direct light of one or more wavelengths onto a skin area; an optical sensor configured to receive the light reflected from the skin area illuminated by the illumination source and generate a corresponding electronic signal; a memory containing computer-readable instructions for: processing the electronic signal to identify one or more properties of the reflected light received by the optical sensor for use in characterizing one or more skin types within the skin area, and automatically characterizing the one or more skin types within the skin area based at least in part on the one or more identified properties of the reflected light; and a processor configured to read the computer-readable instructions from the memory and automatically characterize the one or more skin types within the skin area. Corresponding methods are disclosed.

### CROSS-REFERENCE TO RELATED APPLICATION(S)

This application claims the benefit of and priority to U.S. Provisional Application No. 62/602,463, filed Apr. 25, 2017, which is hereby incorporated herein by reference in its entirety for all purposes.

### TECHNICAL FIELD

The present disclosure relates generally to aesthetic skin treatments, and more particularly, to systems and methods for assessing characteristics of the skin before, during, and/or after an aesthetic treatment.

### BACKGROUND

For many aesthetic and dermatological procedures, it is useful to determine skin characteristics as a guide to therapy. For example, procedures such as, for example, hair and tattoo removal, skin tightening, skin resurfacing, and any other procedure involving application of intense light require evaluation of skin characteristics for determining a suitable light intensity and dosage levels associated with a particular procedure. In particular, many procedures require determination of skin typing according to the established Fitzpatrick Scale, which is a determination of the properties of skin color, reflectivity, and sensitivity to sunburn. Historically, the process of the determination of the Fitzpatrick skin type involves comparison of skin color to a standard color chart and interrogation of the subject to questions regarding sensitivity to burning during exposure to direct sunlight. Such approaches can be subjective, time consuming, and inadequate for detecting variations in skin type across large areas of the body to be treated. Accordingly, there is a need for improved approaches to characterizing skin type.

### SUMMARY

The present disclosure is directed to a system for characterizing skin type. The system, in various embodiments, may comprise an illumination source configured to generate and direct light of one or more wavelengths onto a skin area; an optical sensor configured to receive the light reflected from the skin area illuminated by the illumination source and generate a corresponding electronic signal; a memory containing computer-readable instructions for: processing the electronic signal to identify one or more properties of the reflected light received by the optical sensor for use in characterizing one or more skin types within the skin area, and automatically characterizing the one or more skin types within the skin area based at least in part on the one or more identified properties of the reflected light; and a processor configured to read the computer-readable instructions from the memory and automatically characterize the one or more skin types within the skin area.

The illumination source, in various embodiments, may include one or more light emitting diodes (LEDs), laser diodes, incandescent bulbs, and fluorescent lamps, or any combination thereof. The one or more wavelengths of light generated by the illumination source may include any one or combination of wavelengths on a spectrum between ultraviolet (UV) and near infrared (NIR), inclusive. Additionally or alternatively, the illumination source, in various embodiments, may include a blackbody radiation source and the optical sensor may include one or more spectral filters for selectably filtering one or more wavelengths of reflected light from the skin area illuminated by the blackbody radiation source.

The optical sensor, in various embodiments, may include an image sensor, a charged coupled device (CCD) image sensor, a complementary metal-oxide-semiconductor (CMOS) image sensor, a digital camera, or any combination thereof.

The one or more properties of the reflected light, in various embodiments, may include properties indicative of photo-response by one or more chemical chromophores in the skin area. In some embodiments, the one or more properties of the reflected light may include intensity, color, or a combination thereof.

Skin type, in various embodiments, may include a characterization of one or more properties of the skin that may contribute to the skin's sensitivity and reaction to one or more wavelengths of light, acids, bases, chemicals, or any combination thereof. Automatically characterizing the one or more skin types, in various embodiments, may include evaluating one or more algorithms using, as inputs, measurements of the one or more identified properties. Additionally or alternatively, automatically characterizing the one or more skin types may include evaluating the one or more properties of the reflected light against the Fitzpatrick Scale. The processor, in various embodiments, may automatically characterize the one or more skin types in the skin area in real-time or near real-time.

The processor, in various embodiments, may be further configured to characterize skin type for multiple portions of the skin area, and associate the skin type characterization for each of the multiple portions of the skin area with information concerning a location of each of the multiple portions of the skin area. Additionally, the processor, in some embodiments, may be configured to generate a map or other visual aid for visually presenting variations in the skin type characterizations across the skin area.

The system, in various embodiments, may further include one or more electromagnetic radiation (EMR) sources for generating one or more EMR beams configured for aesthetic or dermatological skin treatment. The processor, in various embodiments, may be further configured to identify one or more adjustments to one or more parameters of the one or more EMR beams based on the one or more skin type characterizations for presentation to an operator, and/or automatically adjust one or more parameters of the one or more EMR beams based on the one or more skin type characterizations.

The system, in various embodiments, may further include an articulable arm for positioning at least the optical sensor of the system. The processor, in various embodiments, may be further configured to associate the one or more skin type characterizations with at least one of a position and orientation of the optical sensor at the time the optical sensor generated the corresponding electronic signal.

In another aspect, the present disclosure is directed to a method for characterizing skin type. The method, in various embodiments, may comprise the steps of: illuminating an area of skin with one or more wavelengths of light; receiving the light reflected from the illuminated skin area and generating a corresponding electronic signal; processing the electronic signal to identify one or more properties of the reflected light for use in characterizing one or more skin types within the skin area, and automatically characterizing the one or more skin types within the skin area based at least in part on the one or more identified properties of the reflected light.

The one or more wavelengths of light, in various embodiments, may include any one or combination of wavelengths on a spectrum between ultraviolet (UV) and near infrared (NIR), inclusive. The one or more properties of the reflected light, in various embodiments, may include properties indicative of photo-response by one or more chemical chromophores in the skin area. In some embodiments, the one or more properties of the reflected light may include intensity, color, or a combination thereof.

Automatically characterizing the one or more skin types, in various embodiments, may include evaluating one or more algorithms using, as inputs, measurements of the one or more identified properties. Additionally or alternatively, automatically characterizing the one or more skin types, in various embodiments, may include characterizing the one or more skin types includes evaluating the one or more properties of the reflected light against the Fitzpatrick Scale. In various embodiments, automatically characterizing the one or more skin types in the skin area may be performed in real-time or near real-time.

The method, in various embodiments, may further include characterizing skin type for multiple portions of the skin area, and associating the skin type characterization for each of the multiple portions of the skin area with information concerning a location of each of the multiple portions of the skin area. In some embodiments, the method may further include generating a map or other visual aid for visually presenting variations in the skin type characterizations across the skin area.

The method, in various embodiments, may further including at least one of: identifying one or more adjustments to one or more parameters of one or more electromagnetic radiation (EMR) beams used for aesthetic or dermatological treatment of the skin area based on the one or more skin type characterizations for presentation to an operator, and automatically adjusting one or more parameters of one or more electromagnetic radiation (EMR) beams used for aesthetic or dermatological treatment of the skin area based on the one or more skin type characterizations.

In various embodiments, an optical sensor may receive the light reflected from the illuminated skin area and generate the corresponding electronic signal, and the method further includes positioning at least the optical sensor of the system using an articulable arm. The method, in some embodiments, may further include associating the one or more skin type characterizations with at least one of a position and orientation of the optical sensor at the time the optical sensor generated the corresponding electronic signal.

### BRIEF DESCRIPTION OF THE DRAWINGS

Illustrative, non-limiting example embodiments will be more clearly understood from the following detailed description taken in conjunction with the accompanying drawings.

FIG. 1 is a block diagram illustrating a multifunction system in accordance with an embodiment of the present invention.

FIG. 2 is a perspective view of electromagnetic radiation emission components of a multifunction system in accordance with an embodiment of the present invention.

FIG. 3 is an interior view of a beam combiner of a multifunction system in accordance with an embodiment of the present invention.

FIG. 4 is a schematic view of power and control electronics of a multifunction system including a plurality of EMR drivers in accordance with an embodiment of the present invention.

FIG. 5 is a perspective view of a cooling system of a multifunction system in accordance with an embodiment of the present invention.

FIG. 6 is a perspective view of a cooling mount of a multifunction system in accordance with an embodiment of the present invention.

FIG. 7 is a perspective view of a refrigeration unit of a cooling system of a multifunction system in accordance with an embodiment of the present invention.

FIG. 8 is a perspective view of a two degree of freedom positioning apparatus in accordance with an embodiment of the present invention.

FIG. 9 is a perspective view of a six degree of freedom positioning apparatus in accordance with an embodiment of the present invention.

FIG. 10 is a schematic view of a subcutaneous temperature prediction system in accordance with an embodiment of the present invention.

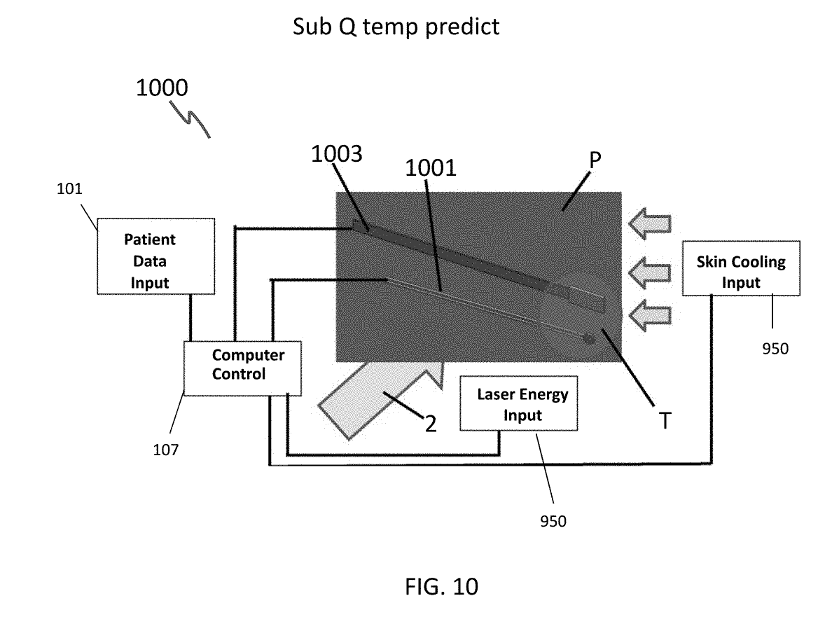

FIG. 11 is a human tissue profile showing expected penetration depth of various EMR wavelengths in accordance with an embodiment of the present invention.

FIG. 12 is a schematic view of a multifunction system including a switching device in accordance with an embodiment of the present invention.

FIG. 13 is a schematic view of a FET circuit of a switching device in accordance with an embodiment of the present invention.

FIG. 14A is a perspective view of a fiber combiner for providing two separate output paths in accordance with an embodiment of the present invention.

FIG. 14B is a detail view of the fiber combiner of FIG. 14A in accordance with an embodiment of the present invention.

FIG. 15 is a cross-sectional view of a device having split, angled EMR beam delivery in accordance with an embodiment of the present invention.

FIG. 16A is a cross-sectional view of a device having beam shaping optics in accordance with an embodiment of the present invention.

FIG. 16B is a cross-sectional view of the device of FIG. 16A having an adjustable optical element in accordance with an embodiment of the present invention.

FIG. 16C is a cross-sectional view of the device of FIG. 16A having an additional optical element in accordance with an embodiment of the present invention.

FIG. 17 is a perspective view of a device having non-contact sensors in accordance with an embodiment of the present invention.

FIG. 18 is a perspective view of an imaging system for determining skin type in accordance with various embodiments.

FIG. 19 is a perspective view of a field of illumination of an imaging system for determining skin type in accordance with various embodiments.

FIG. 20A is a schematic view of a sequentially changing filter behind a black board source in accordance with various embodiments.

FIG. 20B is a graph depicting a blackbody radiation spectrum versus silicon spectral sensitivity in accordance with various embodiments.

FIG. 21 is a block diagram of a system for automatically characterizing skin type in accordance with various embodiments.

FIG. 22 is a block diagram of a system for displaying information concerning skin type of a patient to a dermatologist, clinician, or other person evaluating or performing aesthetic or dermatological treatment of the patient in accordance with various embodiments.

FIG. 23 is a plot illustrating a chromaticity diagram for skin typing in accordance with various embodiments.

FIG. 24 is a perspective view of system for determining skin type and performing aesthetic skin treatment in accordance with an embodiment of the present invention.

### DETAILED DESCRIPTION

Various exemplary embodiments will be described more fully hereinafter with reference to the accompanying drawings, in which some example embodiments are shown. The present disclosure may, however, be embodied in many different forms and should not be construed as limited to the example embodiments set forth herein. Rather, these example embodiments are provided so that this disclosure will be thorough and complete, and will fully convey the scope of the present disclosure to those skilled in the art. In the drawings, the sizes and relative sizes of layers and regions may be exaggerated for clarity. Like numerals refer to like elements throughout.

Unless otherwise defined, all terms, including technical and scientific terms, used herein have the same meaning as commonly understood by one of ordinary skill in the art to which this disclosure belongs. For example, when an element is referred to as being “operatively engaged” with another element, the two elements are engaged in a manner that allows electrical and/or optical communication from one to the other.

### Multifunction Aesthetic Treatment System

Systems and methods for characterizing skin type of the present disclosure, in various embodiments, may be used in conjunction with laser systems for performing aesthetic skin treatments, as later described in more detail. One such laser system, in particular a multifunction aesthetic treatment system 10, is further described herein and in further detail in U.S. patent application Ser. No. 15/820,737, filed Nov. 22, 2017, which is incorporated by reference here in its entirety for all purposes. In particular, in some embodiments, multifunction aesthetic system 10 can include at least two electromagnetic radiation (EMR) sources and a beam combiner for combining electromagnetic radiation beams emitted by the at least two sources. In this manner, the multifunction aesthetic system can emit multiple wavelengths of electromagnetic radiation through a single output device. In some embodiments, the multiple wavelengths can be emitted simultaneously, in alternating pulses, and/or sequentially to permit multiple treatments to be performed by the same multifunction aesthetic system. In some embodiments, the multiple treatments can be performed sequentially, simultaneously, or in alternating fashion.

As used herein, EMR can refer to electromagnetic radiation having any desired wavelength. In particular, EMR generated and/or emitted by embodiments of the present disclosure can be any suitable wavelength, including, for example, visible light, ultraviolet radiation, x-ray radiation, infrared radiation, microwave radiation, radio waves, or combinations thereof.

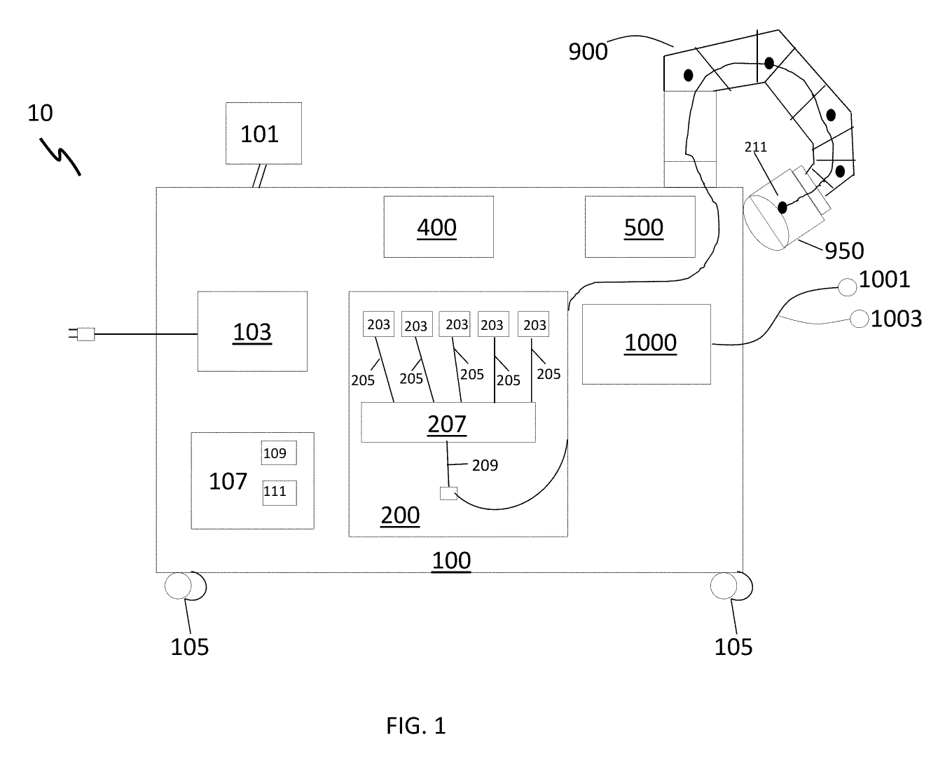

Referring now to FIG. 1, multifunction aesthetic system 10 can be provided for performing a variety of aesthetic procedures in a single medical device. The system 10 can include a housing 100 for housing, retaining, mounting, or engaging components of the system 10. In some embodiments, the housing 10 can be constructed of any suitable material for providing structural support to and protection of components housed, retained, mounted, or engaged in, on, or with the housing 100, including, for example, plastics, polymers, metals, or any other medically compliant material. To the extent that it is desired to move the system 10, for example, from one exam room or operating room to another, the housing 100 can include one or more wheels 105 to provide mobility of the system 10. To the extent that power is required to be delivered to the system 10, the housing 100 can include one or more power cords 103 for engagement with an AC power source such as, for example, a wall outlet.

In some embodiments, the system 10 can include a user interface 101 mounted to the housing 100 for receiving a user input. The user interface 101 can include, for example, an electronic display, a touch-screen monitor, a keyboard, a mouse, any other device or devices capable of receiving input from a user, or combinations thereof. The user input can include, for example, patient data such as height, weight, skin type, age, etc. as well as procedural parameters such as desired beam power, procedure type, wavelength or wavelengths to be applied, pulse duration, treatment duration, beam pattern, etc.

In some embodiments, the system 10 can also include a computing device 107 for receiving and storing the user input from the user interface 101, for storing and executing appropriate procedure protocols according to the user input, for providing control instruction to various components of the system 10, and receiving feedback from the various components of the system 10. The computing device 101 can be any suitable computing device such as, for example, a laptop, a desktop, a server, a smartphone, a tablet, a personal data assistant, or any other suitable computing device having a memory 109 and a processor 111. The memory 109, in some embodiments, can be any suitable memory 109 for storing electronic data, including the user input data and operational data associated with one or more components of the system 10. The memory 109 can include, for example, random access memory (RAM), flash memory, solid state memory, a hard disk, a non-transitory computer readable medium, any other form of electronic memory, or combinations thereof. The processor 111, in some embodiments, can be any processor suitable for receiving user input from the user interface 101, generating commands for operation of one or more system 10 components, executing any software stored in the memory 109, or combinations thereof. The processor, in some embodiments, can include one or more of a microprocessor, an integrated circuit, an application specific integrated circuit, a microcontroller, a field programmable gate array, any other suitable processing device, or combinations thereof.

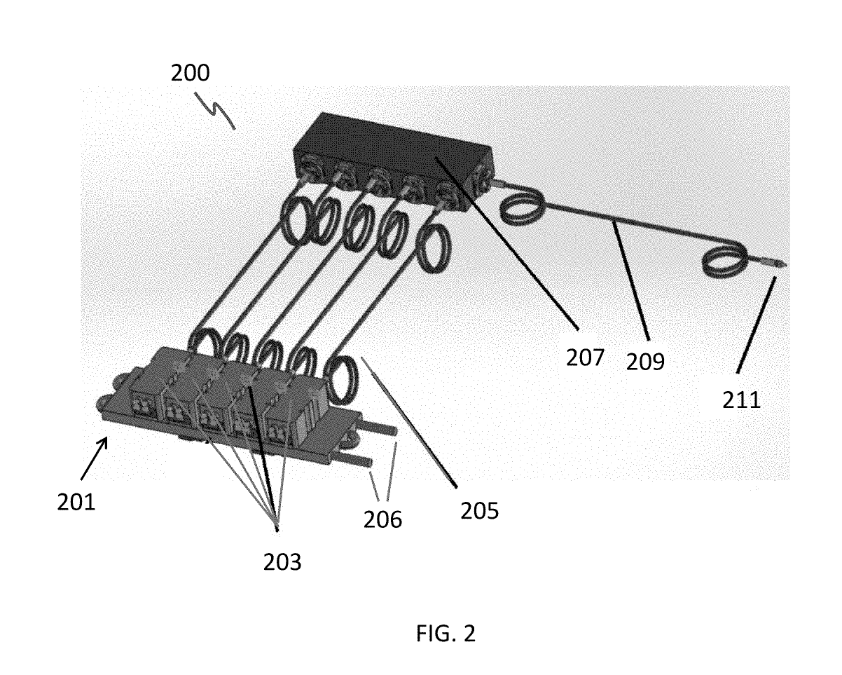

As shown in FIG. 1, the system 10 can also include an electromagnetic array 200. Referring now to FIG. 2, the electromagnetic array 200 can include a mount 201 for mounting a plurality of electromagnetic radiation (EMR) sources thereon. For example, as shown in FIG. 2, the mount 201 includes a plurality of laser sources 203 mounted thereon. The mount 201, in some embodiments, can include any plate, housing, bracket, or other structure for mounting one or more laser sources 203 thereto. As shown in FIG. 2, in some embodiments, the mount 201 can be a cold plate for providing cooling to the laser sources 203 mounted thereto. For example, as illustrated by FIG. 2, the mount 201 can provide first and second coolant ports 201a, 201b for permitting circulation of a coolant through the mount 201. The coolant can then chill the mount 201, thereby providing a heat sink for cooling the laser sources 203 mounted to the mount 201.

In some embodiments, each laser source 203 can be configured to emit EMR at a particular wavelength. For example, in some embodiments, each laser source 203 can emit EMR at a wavelength between about 200 nm to about 4500 nm. However, it will be apparent in view of this disclosure that each laser source 203 can emit EMR at any desired wavelength in accordance with various embodiments. Furthermore, it will be apparent in view of this disclosure that, in addition to laser sources 203, any other source of electromagnetic radiation having any wavelength can be used in accordance with various embodiments. For example, in some embodiments, EMR sources of the system 200 can emit electromagnetic radiation having any suitable wavelength, including, for example, visible light, ultraviolet radiation, x-ray radiation, infrared radiation, microwave radiation, or radio waves. Thus, because each laser source 203 can be configured to emit a different particular wavelength, just one system 10 can produce EMR beams at wavelengths or combinations of wavelengths required for any one of a plurality of procedures having disparate treatment protocol requirements. Accordingly, in some embodiments, the system can include laser sources 203 emitting wavelengths suitable for performing one or more procedures including, for example, but not limited to, fat reduction, body skin tightening, facial skin tightening, skin resurfacing, skin remodeling, vein reduction or removal, facial pigment removal or reduction, hair removal, acne treatment, scar reduction and removal, psoriasis treatment, stretch mark removal, nail fungus treatment, leukoderma treatment, tattoo removal, or combinations thereof.

Some aesthetic procedures may only require a single wavelength. For example, for some fat reduction procedures, a laser source 203 can be provided which is capable of emitting EMR at a wavelength of about 1064 nm (e.g., about 400 nm to about 3000 nm or about 900 nm to about 1100 nm) can be selected for hyperthermia of fat tissue because it exhibits good transmission through the skin, epidermis, and dermis and deposits energy within the fat cells. On the other hand, skin tightening generally requires other wavelengths that exhibit higher absorption in the epidermis and dermis, where the collagen resides. Thus, for example, a wavelength of about 1320 nm (e.g., about 400 nm to about 3000 nm or about 1300 nm to about 1500 nm) can be used for some body skin tightening procedures. These EMR beam wavelengths deposit more energy to the collagen, creating necrosis and eventually skin tightening from new collagen regrowth.

In other examples, such as for some facial pigment reduction or removal procedures and some vein reduction or removal procedures, for example, a laser source capable of emitting EMR at about 532 nm (e.g., about 500 nm to about 650 nm) can be provided.

Additionally, some aesthetic procedures or combinations of procedures may require two or more wavelengths. For example, to combine the fat reduction and body skin tightening procedures discussed above, a first laser source 203 capable of emitting EMR at 1064 nm and a second laser source 203 capable of emitting EMR at 1320 nm can be provided. In another example, for some facial skin tightening procedures, for example, a first laser source 203 capable of emitting EMR at about 1320 nm (e.g., 400 nm to about 3000 nm or about 1300 nm to about 1500 nm) and a second laser source 203 capable of emitting EMR at about 1470 nm (e.g., 400 nm to about 3000 nm or about 1300 nm to about 1500 nm) can be provided.

To provide additional functionality and facilitate ease of maintenance, in some embodiments, the laser sources 203 can be removably mounted to the mount 201 to permit modular replacement of the laser sources 203. Thus, in such modular configurations, individual laser sources 203 can be replaced, for example, to provide additional or different wavelengths or wavelength combinations as needed for particular procedures. However, it will be apparent in view of this disclosure that, in some embodiments, the laser sources 203 can be permanently attached to the mount 201.

The laser sources 203, in some embodiments, can include one or more fiber coupled lasers. For example, in accordance with various embodiments, the laser sources 203 can include one or more fiber coupled diode lasers and/or flashlamp or diode pumped lasers such as Er:YAG, Er,Cr:YSGG, Nd:YAG, Nd:glass; Er:glass, or any other suitable fiber coupled EMR source. In some embodiments, fiber coupled laser sources 203 can be rated as continuous wave (CW) devices operating at 50 W, 100 W, etc. Such CW devices can be operated in a gated mode where the pulse energy is equal to the pulse duration times the power. Therefore, a 100 W diode laser gated to operate for 5 milliseconds will have pulse energy of 500 mJ. In cases where more pulse energy is required but, for example, power supply or cooling capacity limits the average power, fiber coupled laser sources 203 can be configured as a quasi-CW device. Such quasi-CW devices can produce higher power pulses for the same average power draw by operating at a lower pulse frequency rate. In some embodiments, a quasi-CW device can produce pulses having up to 10 times the average power draw. Thus, for example, a 1000 W/100 W quasi-CW diode would be capable of pulsed operation at 5 milliseconds with 5 Joules per pulse, but limited to one tenth the pulse frequency of a CW laser.

In some embodiments, at least one of the laser sources 203 can include a fiber coupled diode laser. Such laser systems can advantageously operate at efficiencies exceeding 50%, are relatively small in size, draw relatively low power, and exhibit wide wavelength diversity. Fiber coupled diode lasers can, for example, be driven by less than 2.0 volts DC to produce an output of 10 kW or more. Furthermore, such laser sources 203 can be small and lightweight, with the module weighing about 500 grams per 1 kW. In one embodiment, at least one of the laser sources 203 can be a 75 W fiber coupled diode having a size of about 8×4×3 cm (less than 100 cm³). In some embodiments, such laser sources 203 can be used to perform an aesthetic procedure while drawing less than 100 Watts of power. Such low power draw can, in some embodiments, reduce the amount of cooling required, permitting smaller, quieter, more efficient cooling systems.

The compliance voltage for nearly all diodes of interest is slightly less than 2.0 VDC. Packaging and differing bias voltage configurations can be applied to result in a common higher voltage which then allows a lower drive current. For example, a typical 50 W diode driven at 2.0 VDC can require a minimum threshold current of 8 amps to 12 amps and can require more than 60 to 70 amps to produce a desired power level. Such high current necessitates heavy gauge wiring such as #6 or #8 gauge wires to avoid voltage drop, preserve system reliability, and minimize Joule heating. To reduce the required current supply and wiring size, in some embodiments, the diode of each fiber coupled diode laser source 203 can be configured to operate with a common compliance voltage such as, for example, 20 VDC or 25 VDC, with a drive current controlled to match the laser selected and the required output power. By increasing the common compliance voltage to 20 or 25 VDC, the maximum drive current required to operate each laser source 203 can be limited to about 10 amps or less for most aesthetic procedures. By reducing required current, smaller gauge wiring can be used to improve reliability. In some embodiments, such an approach permits use of a single power supply to drive more than one of the laser sources 203 by manifolding the power supply into connections with multiple EMR sources. Thus, for example, in embodiments where only one laser is operated at a time, then the system 10 may be provided with only one power supply.

Typical diode packaging employs semiconductor bars with compliance voltages near 2.0 VDC, where threshold currents are in the 8 to 12 amperage range. To reach significant power levels, such diodes can operate as high as 70 amps. The associated problem with these voltage drops and joule heating (I²*R) adds to reliability concerns. However, partial diode bars (i.e., diode bars having a shorter length than a standard 2.0 VDC diode bar) typically require less current proportional to the bar fraction. Thus, by using partial diode bars connected in series, delivering lower current but at a higher voltage for activating each of the partial diodes, required current can be reduced while power is maintained.

In some embodiments, at least one of the laser sources 203 can include a flashlamp or diode pumped laser. For example, many aesthetic skin treatments require application of EMR having a wavelength near 3000 nm, such as, for example, wavelengths greater than 2500 nm. Such wavelengths are typically produced by flashlamp or diode pumped solid state laser devices such as Er:YAG, which produces EMR having a wavelength of about 2940 nm or Er:YSGG, which produces EMR having a wavelength of about 2790 nm. However, although shown and described herein with reference to fiber coupled diode lasers and flashlamp or diode pumped lasers, it will be apparent in view of this disclosure that any suitable type of EMR source capable of being coupled to a fiber optic output cable can be used in accordance with various embodiments. In some embodiments, laser sources 203 including the flashlamp or diode pumped solid state laser devices can also be configured to operate at the common compliance voltage as explained above with reference to the fiber coupled diode lasers. Thus the system 10, in some embodiments, can use the common power source as discussed above with reference to the fiber coupled diode lasers.

Still referring to FIG. 2 the electromagnetic array 200 can also include a fiber optic relay cable 205 coupled to each laser source 203 for transmitting or relaying the EMR (also referred to as “EMR energy” or “beam”) emitted by the respective laser source 203. In general, each fiber optic relay cable 205 can be constructed of any fiber optic material capable of transmitting EMR having a wavelength emitted by each respective laser source 203. In some embodiments, each fiber optic relay cable 205 can be constructed of, for example, low-OH silica fiber core cables, which transmit wavelengths in a range of about 200 nm to about 2400 nm, Zirconium Fluoride (ZrF4) and/or high purity Chalcogenide glass cables, which transmit wavelengths in a range of about 285 nm to about 4500 nm, or sapphire cables, which transmit wavelengths in a range of about 170 nm to about 5500 nm.

In some embodiments, the fiber optic relay cables 205 can be mated to the laser sources 203 by a fiber optic connector such as, for example, a SMA 905 connector or any other suitable connector. For each of the fiber optic relay cables, the fiber core diameter can be driven by the coupling efficiency of the diode driver and the required power. For example, in CW operation, in one embodiment, for near infrared wavelength ranges, the core diameter can be determined by an energy density limit in the cable of about 1.4 MW/cm² to provide a reliable relay. This reliability limit on the fiber predicts that a 100-micron core diameter can handle up to 85 W and a 400 micron core diameter can be used up to 1300 W. Shorter wavelengths typically scale to lower power limits. Additionally, for pulsed operation where the pulse duration is less than one (1) microsecond (1×10⁻⁶ seconds), fiber damage is not thermal but caused by dielectric breakdown and occurs at lower levels proportional to the pulse duration. That is, although average power is low enough to prevent overheating of the fiber, the power delivered during a pulse duration of less than one (1) microsecond can cause breakdown of the dielectric materials of the fiber. More generally, by selecting the proper fiber core diameter and connectors capable of handling maximum expected power loadings, safe and reliable routing of the EMR power generated by the laser sources 203 is possible.

Still referring to FIG. 2, the system can also include a beam combiner 207 for combining the EMR beams produced by each laser source 203 and transmitted by each relay cable 205 into a single output. Generally, the beam combiner 207 can be any device or system capable of combining several EMR beams of different wavelengths into one output. For example, in some embodiments, the beam combiner can include, for example, fiber switching devices, free-space fiber combiners, butt-coupled combiners, tapered fibers, bundled fibers, and fused fibers.

For example, free space combiners can be packaged with mirrors and gratings to fold separate beams into one fiber. Butt-coupled fiber combiners can mate smaller core fibers into a larger core output cable. For butt-coupled fiber combiners, the smaller fibers are stripped to their cladding and packaged as close to each other as possible, for example, in a circular footprint. The polished fiber ends can be mated (butt-coupled) to a larger fiber core with a diameter greater than the multiple fiber footprint. Tapered fibers can be used to reduce the core diameter of the combined fibers. That is, tapered fibers can be stretched such that the diameter of each tapered fiber is reduced to permit a higher packaging density for fiber coupling. Fiber fusing can be used to mate multiple fibers together by stripping the fibers and bundling them into a close-packed cross-section. The fibers can then be heated and melted to fuse into a single output fiber. Bundled fiber cables can also be used to route multiple sources into one output path. Bundled fibers, in general, can be larger diameter fiber cables formed from many small, individual fibers closely packed within the cable.

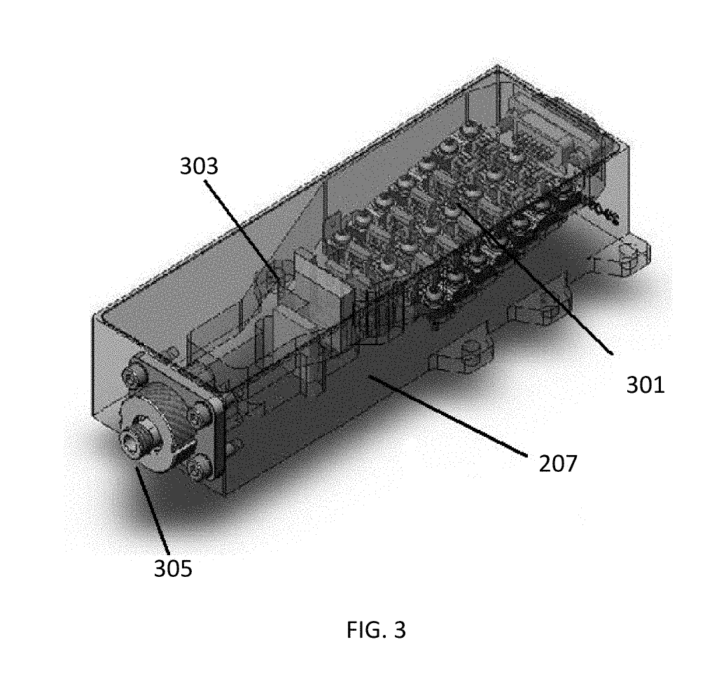

Additionally, as shown in FIG. 3, in some embodiments, the beam combiner 207 can include a high brightness/low cost fiber coupling package such as the device produced for nLight Corporation under NASA SBIR program 05-II S6.02-8619. The device can include multiple diodes 301 all coupled into a single core fiber output port 305. The beam combining optics 303 can be configured to converge each of the individual diode 301 outputs into a common optical path. The beam combiner can then route the converged outputs to an output port 305 (e.g., a SMA 905 connector). The beam combiner 207, in some embodiments, can be configured to combine diverse beam wavelengths for beam powers ranging from a few Watts to more than 10 kW.

In such embodiments, because only the laser sources 203 producing the desired wavelengths are activated at any time, the beam combiner 207 can be a passive device, rather than an active fiber switch. Having a passive device also helps in defining the power limits for the fibers, where the limit in watts for the fibers can be matched to the highest power laser source 203 available where only a single laser source 203 is active at a time, rather than a sum from each laser source 203. To the extent that multiple laser sources 203 are activated simultaneously, the power limit of the combined fibers must be equivalent to at least the sum of the power required to operate each active laser source 203. Alternatively, in some embodiments, the beam combiner 207 can also include one or more fiber switches to selectively output particular wavelengths.

The beam combiner 207 can then output the combined beam to a common output cable 209 coupled to the beam combiner 207 for transmitting or relaying the EMR (also referred to as “treatment energy” or “beam”) combined in the beam combiner 207. Advantageously, the common output cable 209 can permit the different beams produced by the laser sources 203 to be emitted through a single optical device. In particular, by combining or directing the beams in the beam combiner 207 to the common output cable 209, a single optical device of the system 10 can emit beams of different wavelengths simultaneously, sequentially, or in an alternating pulsed pattern. Thus, advantageously, in some embodiments, two or more treatment procedures can be performed simultaneously, contemporaneously, or immediately sequentially to improve patient outcomes and to reduce a number of patient follow up procedures.

In some embodiments, the fiber optic output cable 209 can be, but is not limited to, substantially similar to fiber optic relay cables 205. More generally, the fiber optic output cable 209 can be any fiber optic cable capable of transmitting the combined beam emitted by the beam combiner 207 to a fiber optic output. In accordance with various embodiments, the output cable 209 can be formed as a single fiber, can be formed as a plurality of smaller, bundled fibers, or can be formed as two or more closely packed individual fibers for separately transmitting two or more distinct beams having different wavelengths.

More generally, although the relay cables 205 and the output cable 209 are shown herein as being fiber optic cables, it will be apparent in view of this disclosure that any optical pathway capable of directing or transmitting EMR from one or more EMR sources to the beam combiner 207 and from the beam combiner 207 to the treatment area can be used in accordance with various embodiments. For example, in some embodiments, the pathways can be constructed of a series of mirrors for directing the EMR beams.

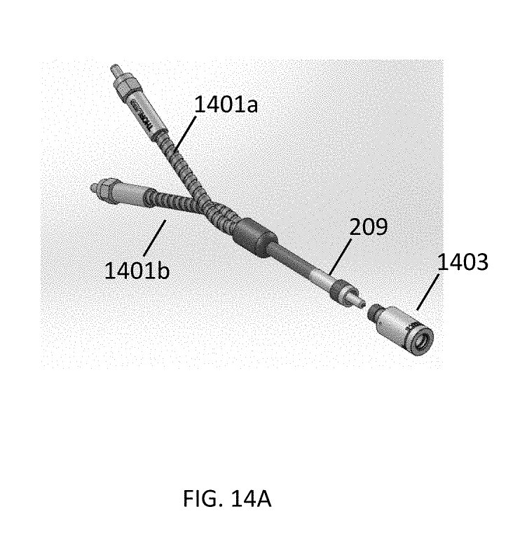

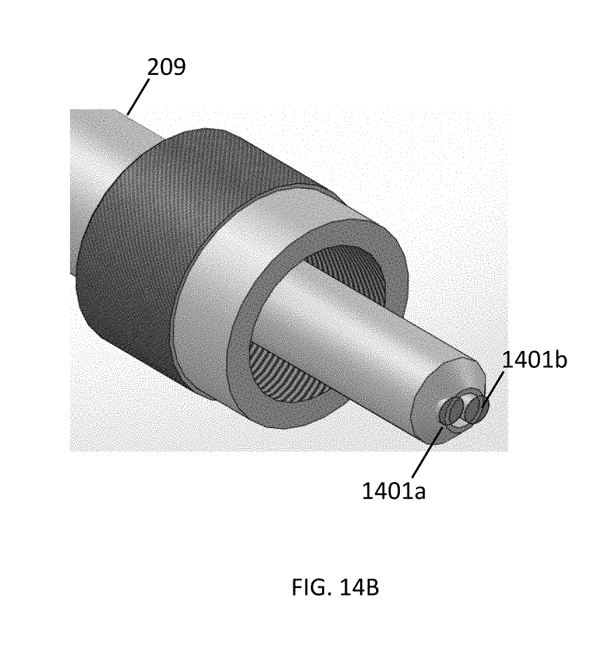

For example, as shown in FIG. 14A, in order to route two separate beams from two distinct EMR sources to a single delivery device (e.g., a hand piece, robotic head, beam shaping optics) 1403, two individual fiber cores 1401a, 1401b can be combined to form a common output cable 209 to direct a beam from each active laser source 203 into a single output fiber connector 211. Referring now to FIG. 14B, because the fiber cores 1401a, 1401b of the common output cable 209 are adjacent and positioned near a center of an optical axis of one or more beam shaping components 1403, the beam shaping components 1403 can produce EMR beam outputs from either or both laser sources 203 with only a slight angular deviation from the true optical axis, the deviation having a negligible effect on beam shape and orientation.

In some embodiments, the fiber optic output cable 209 can also include a fitting 211 positioned at one end thereof for engagement with a device such as a hand piece, robotic head, or other emitter.

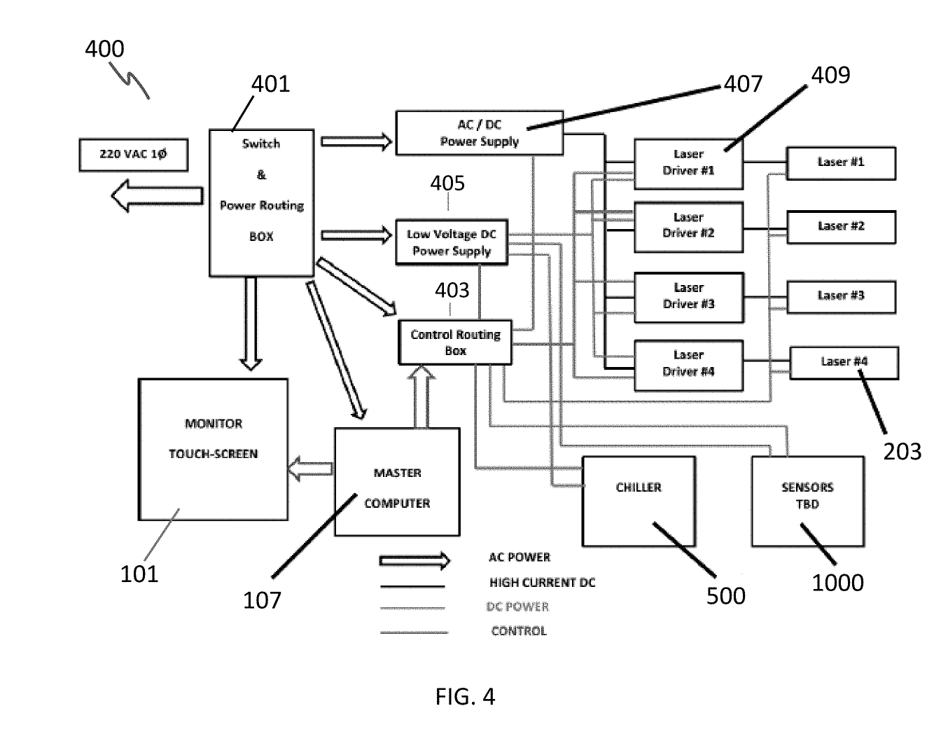

As shown in FIG. 1, in some embodiments, the system 10 can include power and control electronics 400 for powering and controlling various components of the system 10. Referring now to FIG. 4, in some embodiments, power and control electronics 400 can include a switch and power box 401 for receiving AC electrical power from the power cord 103 and distributing AC electrical power to various components as required for operation of the system 10.

The power and control electronics 400 can also include a controller 403, powered by the AC electrical power, in electronic communication with the computing device 107 to command one or more additional components of the system 400 to perform one or more directed operations to execute an aesthetic procedure.

The power and control electronics 400 can also include a low voltage ADC 405 for converting AC power from the power box 401 into low voltage DC power for operating one or more additional components of the power and control electronics 400. The low voltage ADC 405 can include any suitable ADC, including, for example, a direct conversion ADC, successive approximation ADC, ramp compare ADC, Wilkinson ADC, integrating ADC, delta encoded ADC, pipelined ADC, sigma delta ADC, time interleaved ADC, intermediate FM stage ADC, any other suitable ADC, or combinations thereof.

The system can also include a high voltage ADC 407 for converting AC power from the power box 401 into high voltage DC power for operating one or more additional components of the power and control electronics 400. The high voltage ADC 407 can include any suitable ADC, including, for example, a direct conversion ADC, successive approximation ADC, ramp compare ADC, Wilkinson ADC, integrating ADC, delta encoded ADC, pipelined ADC, sigma delta ADC, time interleaved ADC, intermediate FM stage ADC, any other suitable ADC, or combinations thereof.

The power and control electronics 400 can also include a plurality of diode drivers 409 for delivering drive current to the laser sources 203. The diode drivers 409, in some embodiments, can, for example, be semiconductor devices configured to pass a high current through a junction region between an n-type semiconductor and a p-type semiconductor. In such configurations, electrons produced by the n-type semiconductor in the presence of a current source such as DC power supply 407 can result in production of photons upon encountering holes of the p-type semiconductor. The photons can oscillate within the junction region, resulting in an optical gain in the junction region. When the current delivered to the semiconductor device exceeds a threshold current, the optical gain can exceed a threshold intensity, causing the photons to exit the junction region as a beam of laser light. In general, after reaching the threshold current, the laser output increases in power density (intensity) linearly in proportion to an increase in the input current. Furthermore, in some embodiments, the diode drivers 409 can also include regulators for controlling current input and one or more protective features such as, for example reverse current blocking and electrical spike suppression features.

In some embodiments, a single DC power supply 407 can be used for multiple diode drivers if the required compliance voltage for each driver 409/laser source 203 pair is the same and within the limits of the chosen diode driver. Sufficient current capability of the DC power supply 407 to operate the number of simultaneously driven driver 409/laser source 203 pairs is required. Advantageously, no special switching is required between the DC power supply 407 and the driver 409 or driver 409 and laser source 203. The DC power supply 407, in some embodiments, can be parallel connected to each driver 409. This presents an option for multiplexing the main power supply to the multiple laser sources 203.

In such embodiments, each of the diode drivers 409, when activated, can directly drive a single laser source 203 to produce a beam having a particular wavelength as discussed above with reference to FIG. 2. Thus, in some embodiments, one driver 409/laser source 203 pair can be activated for aesthetic procedures requiring a single wavelength EMR beam for treatment. Alternatively, in some embodiments, multiple driver 409/laser source 203 pairs can be activated any of simultaneously, sequentially, or in an alternating pulsed pattern to provide two or more wavelengths as required for a particular treatment and/or to combine or expedite treatments.

Referring again to FIG. 1, the system 10 can also include one or more cooling systems 500 for removing heat produced by the electromagnetic array 200 and the power and control electronics 400 and for delivering cold air for cooling of a patient's skin during a procedure. In general, cooling requirements are primarily dependent on heat generated by the electromagnetic array 200. For example, for a system operating a 100 W EMR source in a small package with an efficiency of about 50%, the cooling capacity can be as low as 200 watts.

Such heat is typically dissipated by one or more of forced air (e.g. fan) cooling, thermoelectric cooling, flowing coolant directly through the electromagnetic array 200, or a cooling plate. However, in general, forced air cooling is noisy and not efficient, thermoelectric coolers have relatively poor efficiency, requiring excessive heat dissipation at a heat sink. Other devices employ circulating coolant directly in the electromagnetic array 200, which can result in difficult maintenance and places a circulating fluid in close proximity to delicate optics, semiconductors, and high current. By contrast, baseplate cooling to cold plate is efficient, safe, quiet, and compact. Large cold plates can accommodate multiple EMR source heads and drive electronics. In some embodiments, several cold plates can be connected in series to the master circulating chiller. In some embodiments, one or more additional master circulating chillers can be provided as required to accommodate different cooling temperature requirements.

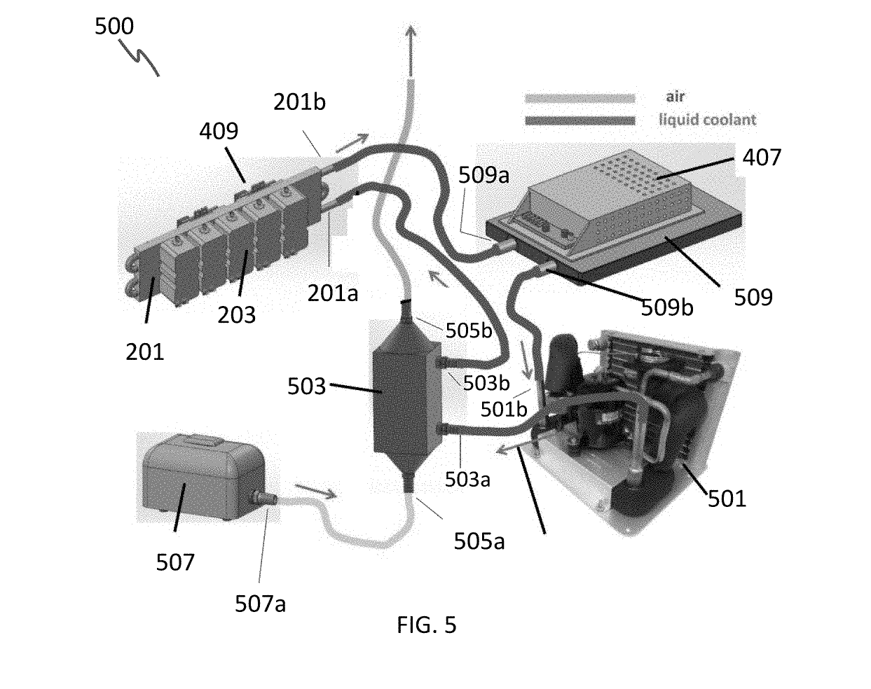

As shown in FIG. 5, the cooling system 500 can include a refrigeration unit 501 such as a refrigerated heat exchanger, thermoelectric cooler, cold water heat exchanger, any other suitable cooling device, or combinations thereof. In some embodiments, a coolant output 501a can exit refrigerated coolant from the refrigeration unit 501. The coolant can then be routed through multiple devices to provide cooling and remove heat before being directed to a coolant return 501b for further refrigeration. Although shown having a single refrigeration unit 501 herein, it will be apparent in view of this disclosure that, in some embodiments, the cooling system 500 can include one or more additional independent refrigeration units 501 to cool various components at different temperatures. For example, in some embodiments, a first refrigeration unit can provide coolant at a temperature of about 0° C. to about 5° C. to chill cooling air for impingement on a patient during a procedure and a second refrigeration unit can provide coolant at a temperature of about 20° C. to about 25° C. to cool the electromagnetic array 200 without generating condensation, which could damage the laser sources 203. It will still further be apparent in view of this disclosure that, in some embodiments, the refrigeration unit 501 and/or the cooling system 500 can be provided with a temperature adjustment feature for permitting responsive adjustment of the coolant temperature depending on operational conditions and/or sensor feedback as needed to maintain therapeutically acceptable temperatures in the treatment area consistent with procedure requirements and to maintain operationally acceptable temperatures within the system 10 consistent with equipment requirements.

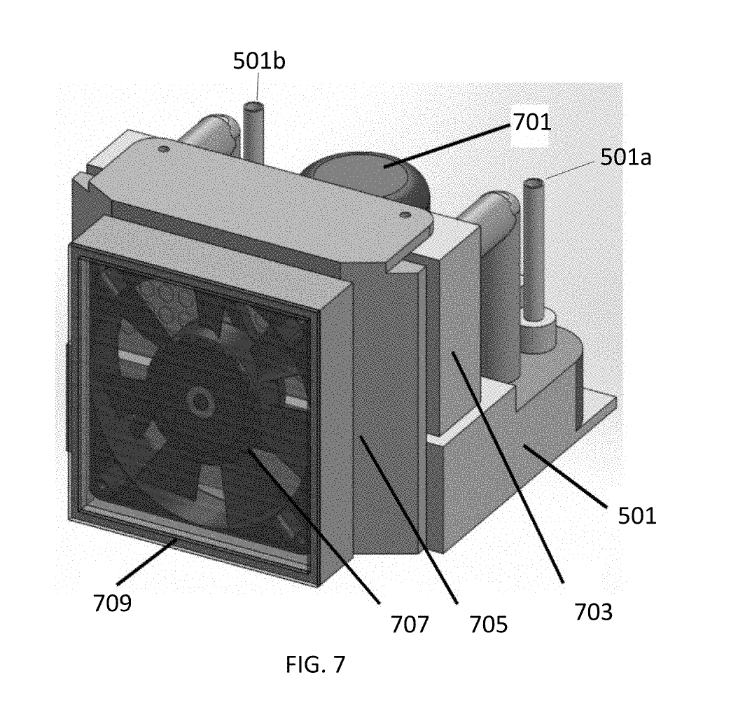

Referring now to FIG. 7, the refrigeration unit 501 can also include a compressor 701, a condenser 703, and an evaporator (not shown). The refrigeration unit 501 can provide forced convection cooling of the condenser 703 through a plenum 705 using a fan 707. In some embodiments, to improve air quality, the plenum 705 and fan 707 can include a HEPA filter 709 to capture particles, bacteria, and viruses, thereby preventing circulation of such particles, bacteria, and viruses through air surrounding the system 10.

In some embodiments, the coolant can be directed to a coolant inlet 503a of a heat exchanger 503, flowed through the heat exchanger 503, and exited from the heat exchanger 503 via coolant outlet 503b. The heat exchanger 503 can be any suitable device for cooling air or other gasses driven through the heat exchanger 503 via gas inlet 505a and exited via gas outlet 505b. The air or gas flowing in the heat exchanger 503, in some embodiments, can be used for cooling the skin of a patient during a procedure. For example, in some embodiments, the air or gas can cool the patient skin to a target temperature in the range of 15 to 20° C. via a gas impingement cooling of the skin during the procedure in order to maintain a therapeutically acceptable temperature range.

In some embodiments, the air or gas can be driven through the heat exchanger 503 by a pump 507. The pump 507, in some embodiments, can be any suitable device capable of driving the gas through the heat exchanger 503 and onward to a jet impingement nozzle (not shown). In some embodiments, in order to maintain a therapeutically acceptable temperature at the treatment area (e.g., a patient's skin), the pump 507 can be in electronic communication with the controller 403 to receive instructions from the controller for adjusting a flow rate of the cooling air or gas responsive to feedback from one or more temperature sensors monitoring the treatment area.

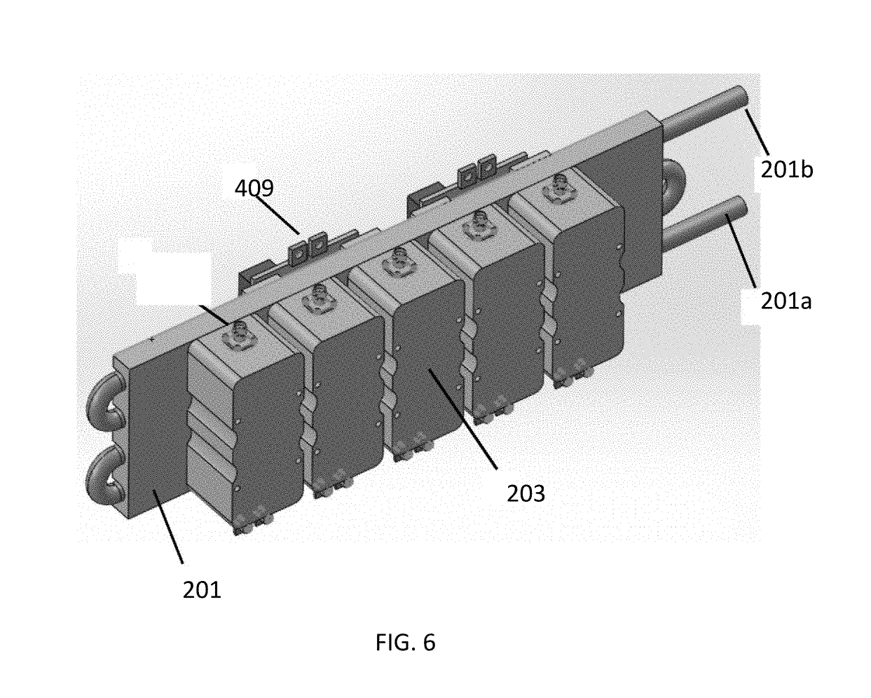

The cooling system 500, in some embodiments, can also route the coolant from the coolant outlet 503b of the heat exchanger 503 to a first coolant port 201a of a mount 201 as described above with reference to FIG. 2. The coolant can chill the mount 201, thereby providing a heat sink for cooling the laser sources 203 mounted to the mount 201. As shown with greater detail in FIG. 6, in some embodiments, the mount 201 can be a cold plate for cooling the laser sources 203 mounted thereto. In some embodiments, the mount 201 can also include one or more of the diode drivers 409 mounted thereto. In such embodiments, the cold plate mount 201 can advantageously cool both the diode drivers 409 and the laser sources 203 with a single cooling mechanism. Although the mount 201 cooling plate is shown herein as being sized for five laser sources 203 and two diode drivers 409, it will be apparent in view of this disclosure that the mount 201 can be sized to accommodate any number or combination of laser sources 203 and diode drivers 409.

Referring again to FIG. 5, the coolant can be exited from the mount 201 via a second coolant port 201b and routed to a coolant input 509a of a baseplate 509 of the DC power supply 407 to provide cooling to the DC power supply 407. The coolant can be exited from the baseplate 509 via a coolant output 509b of the baseplate 509 and routed to the coolant return 501b of the refrigeration unit 501.

In various embodiments, system 10 may additionally or alternatively include a system for cooling the skin via impingement cooling as described in more detail in U.S. patent application Ser. No. 15/820,699, filed Nov. 22, 2017, which is hereby incorporated by reference in its entirety for all purposes.

Referring again to FIG. 1, the system 10 can also include one or more positioning apparatus 900 in accordance with various embodiments for permitting movement, control, and positioning of a device 950 coupled to the common output cable 209. In general, aesthetic EMR devices apply EMR energy with stationary or manually manipulated devices. Thus, the application of the heat energy is typically limited to small fixed areas in the case of stationary devices or, in the case of manually manipulated devices, a relatively uncontrolled and nonuniform dosage of total energy. Accordingly, in some embodiments, the positioning apparatus 900 can provide a multi-axis, computer controlled mechanism for controlled movement, orientation, and positioning of the device 950 used for emitting the EMR beams for treatment. In some embodiments, such positioning apparatus 900 can provide movement over a predefined treatment zone. In some embodiments, the computer control provides improved control and movement over stationary or manually operated systems. In particular, computer control can provide for scanning the device 950 across large areas during treatment to provide uniform heating of the target treatment area. Furthermore, the treatment pattern can be modified to any shape desired for treatment. For example, treatment patterns can be programmed to avoid existing scar tissue or the belly button area, where no target fat exists.

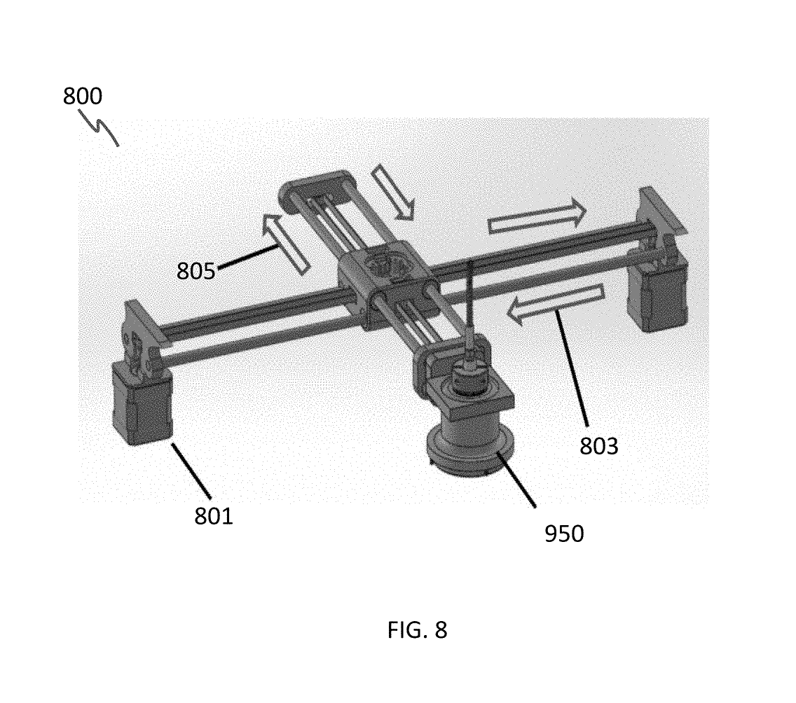

In order to provide desired coverage of an area to be treated and permit proper positioning of the device 950, the positioning apparatus 900 can be provided with any number of degrees of freedom for movement of the device 950. For example, in some cases a treatment process can employ only one DOF and move the device 950 back and forth over the treatment area. As shown in FIG. 8, in some embodiments having a substantially planar target treatment area, the positioning apparatus can be a two degree of freedom control device 800 having a first rail 803 for providing movement along an x-axis of the device 800 and a second rail 805 for providing movement along a y-axis of the device 800.

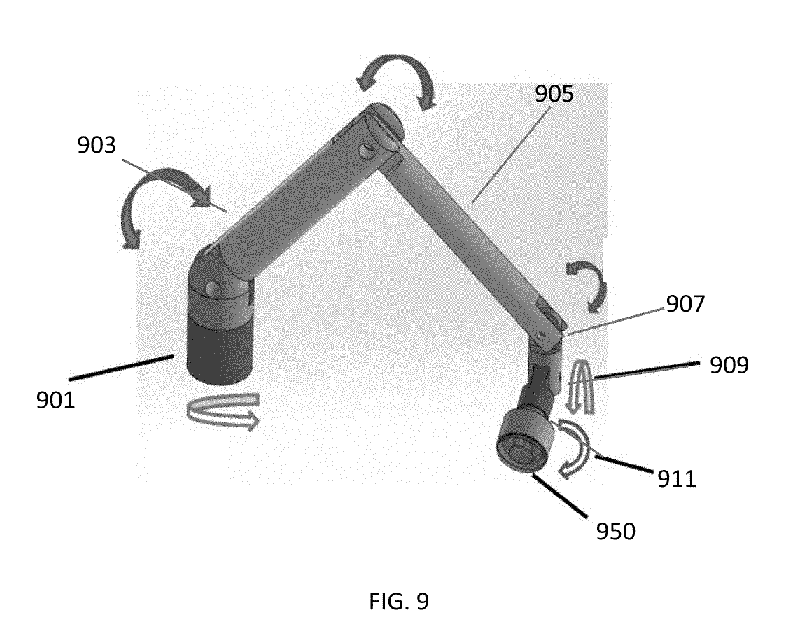

Referring now to FIG. 9, in some embodiments, the positioning apparatus 900 can be a six degree of freedom robotic arm. The positioning apparatus 900 can include, for example, a rotatable base 901 providing a first degree of freedom of rotation of the positioning apparatus 900. The rotatable base 901 can be pivotably engaged with a first segment 903 to provide a second degree of freedom. The first segment 903 can be pivotably engaged with a second segment 905 to provide a third degree of freedom. The second segment 905 can be pivotably engaged with a third segment 907 to provide a fourth degree of freedom. The third segment 907 can be pivotably engaged with a fourth segment 909 to provide a fifth degree of freedom. The fourth segment 909 includes a rotatable portion 911 for rotating the device 950. In general, the rotatable base 901 can be engaged with the housing 100 of the system 10 or can be attached to a separate platform for positioning nearer the target treatment area. The six degrees of freedom of the positioning apparatus 900 can advantageously be used to follow the targeted patient's body shape and match the treatment zone desired.

Such positioning apparatus 900 can be important in various procedures such as, for example, in the case of subcutaneous fat reduction, where deposition of heat into the subcutaneous fat requires reaching and maintaining a therapeutically acceptable temperature range such as, for example, about 40° C. to about 48° C. over a period of time. In particular, in some embodiments, lower temperatures have no fat reduction benefit and higher temperatures can cause severe necrosis, cell damage, and scarring. Conventional devices modulate or cycle the power off and on to maintain this temperature range. However, the low thermal conductivity of fat makes EMR source on/off cycle times compatible with a scanning or moving the device during treatment to cover larger treatment areas and to avoid overheating of the treated tissue. Thus, the positioning apparatus 900 can be programmed to control the device 950 to follow the targeted patient's body shape and match the treatment zone desired. In such embodiments, the heat energy delivered, the treatment area, the dwell time for energy on and the heat source return time to maintain the target temperature are factors that can be used to determine the overall treatment protocol. Patient information, sensors, and feedback can also all be used to maintain a uniform heating over the entire treatment site by scanning the energy delivery module in such a fashion as to cover the entire site. However, it will be apparent in view of this disclosure that, in some embodiments, the system 10 may not include a positioning apparatus 900 and that the device 950 can instead be connected to the housing by the fiber output 209 and/or a cooling air source for manual operation and positioning. It will still further be apparent in view of this disclosure that, in some embodiments, the system 10 may include both a device 950 for use with the positioning apparatus 900 and a manually operated and positioned device 950 for use as required by a particular procedure. For example, the manually operated and positioned device 950 can be used where desired.

Furthermore, sensors 1000 and corresponding sensor feedback can be monitored in real time by the computing device 107 to permit the computing device 107 to reactively instruct (e.g., via controller 403) the positioning apparatus 900 to reposition the device 950. For example, in some embodiments, if the sensors 1000 detect that skin temperature is too high, the computing system 107 can instruct the positioning apparatus 900 to move the device 950 to a new location and/or to scan faster during treatment to reduce dwell time in one area and prevent overheating. In some embodiments, the if the sensors 1000 detect that skin temperature is too low, the computing system 107 can instruct the positioning apparatus 900 to increase a distance or spacing between the device 950 and the target surface to reduce the effects of cooling air flowing through the device 950. Still further, sensors 1000 can be included to detect a position of the device 950 relative to the surface to be treated. In such embodiments, the positioning apparatus 900 can responsively adjust a position or orientation of the device 950 relative to the surface to be treated according to the sensor 1000 feedback. For example, in some embodiments, the positioning apparatus 900 can maintain a prescribed separation height between the device 950 and the surface to be treated.

Numerical simulation modeling for an EMR source in the near-infrared where transmission to the subcutaneous fat is achieved shows that for 1.5 watts per centimeter squared over a 2×2 inch area, the adipose tissue at 12 mm depth reaches 47° C. within 50 seconds. This sample model also included controlled cooling of the skin at 30° C. Simulations show that, without cooling the skin surface would reach an unacceptable temperature of more than 57° C. In this case, the model also shows how the adipose tissue's temperature will decay with time. This model indicates that the patient can be treated in one zone for 50 seconds, after which the robotic control moves the energy source to the next zone for another 50 seconds. This can be repeated to multiple zones, only requiring return to the initial zone before its temperature falls too far below the target temperature range of 40 to 48° C. for efficient hyperthermia apoptosis. Additional modeling studies show that the second treatment duration requires less time to reach the 48° C. temperature and that the reduction in required reheat time is asymptotic.

It is important to note that this model is an example based on defined tissue characteristics. However, dwell times and reheat cycles may need to be adjusted on a case by case basis based on, for example, patient skin type, patient characteristics, wavelength, cooling characteristics, etc. Additionally, it will be apparent in view of this disclosure that the treatment does not need to target 48° C. and can instead target a lower temperature within a procedure-specific range. For example, the treatment can be successful with lower target temperatures, such as 44° C. In each case, the patient type and treatment time can be adjusted to a range of target temperatures. Additionally, it will be apparent in view of this disclosure that, in some embodiments, the temperature can be permitted to fall below the minimum effective temperature of 40° C. for short periods of time with reheating applied to raise the temperature back into the hyperthermia apoptosis targeted range. The application of computer control with the appropriate input parameters allows an efficient and optimized treatment protocol.

A pattern may be scanned in which the energy source returns to the initial treatment site in a time equal to the expected decay time of the temperature. Since reheating to the target temperature requires less time on the second pass, the energy source may be moved at a faster rate on the second pass over tissues. Energy source scanning patterns may be optimized for treatment of a maximum area in a minimum time, and will depend upon patient anatomy and tissue parameters. Scan rates and treatment patterns may be modified in real time based upon measured skin temperatures and heat flux and predicted subcutaneous tissue temperature. Energy source power may be modulated during movement of the energy source to further optimize treatment.

Referring again to FIG. 1, the device 950, in some embodiments, can be configured to emit the combined beam emitted by the beam combiner 207 and received via the fiber output 209 for treatment of the patient. In some embodiments, one or more devices 950 can be interchangeably engageable with the fitting 211 of the fiber optic output cable 209. In general, the device 950 can include mirrors, beam shaping optics or any other appropriate optical elements. For example, the fiber output can be emitted directly on the patient or mated to a collimating device. In a similar fashion, two or more EMR beams can be combined in free space using mirrors and beam splitting optics. The desired beam shape or pattern on the patient can be modified with an optical element, which can be a lens, lens array, a diffractive beam shaper, or any engineered diffusing device. The resulting beam shape can match the desired treatment pattern. In some embodiments, the output beam can be adjusted to match the desired beam diameter, power level, and be collimated, diverging, or converging. As stated above, one or more of the laser sources 203 can be operated simultaneously, alternately, or in sequences. This can be controlled by the input to each laser source 203 since the fiber cables and routing optics are passive devices. EMR beam switches or interlocks can be included as required for safety and regulation compliance. In some embodiments, the device 950 can also include a distance sensor for providing feedback to the computer 107 for adjusting positioning by the positioning apparatus 900.

Additionally, although shown in FIG. 1 and described herein as being mounted and/or coupled to the positioning apparatus 900, it will be apparent in view of this disclosure that, in some embodiments, the device 950 may, in some embodiments, be used as a manual hand piece. In such embodiments, the device 950 may not be coupled to any positioning apparatus and instead can be coupled to the housing 100 only by the fiber output 209 and/or a cooling air supply for permitting manual operation and positioning of the device 950.

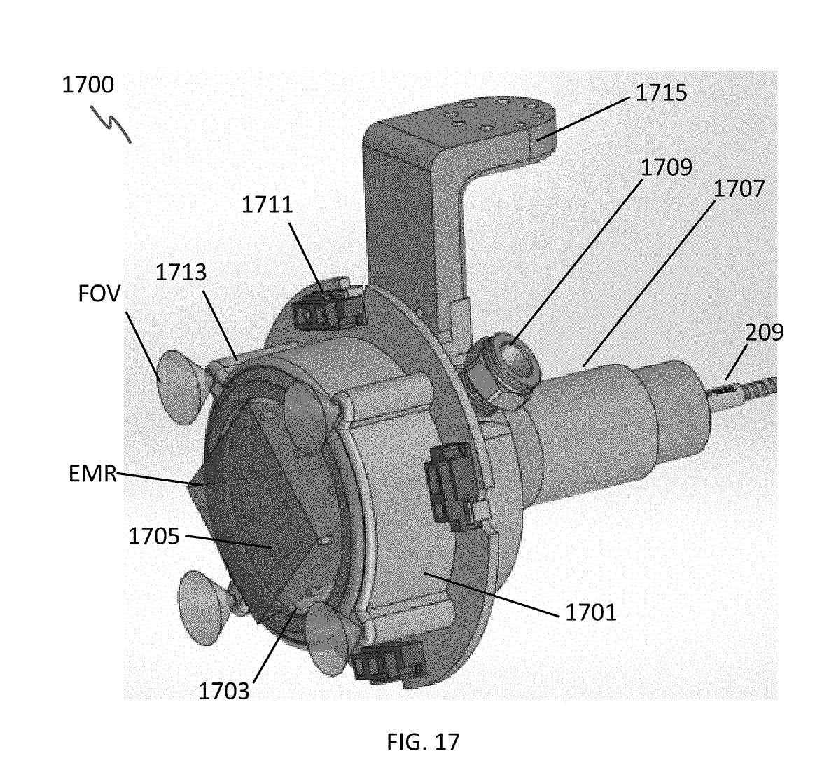

Referring now to FIG. 17, a device 1700 is configured for emitting the EMR beam received via the fiber output 209 for treatment of the patient without contacting the treatment area. In particular, the device 1700 can be configured to direct the EMR beam onto the treatment area, direct cooling airflow onto the treatment area, and provide sensor feedback associated with the treatment area to the controller 403 without making contact with the treatment area.

To that end, the device 1700 can include a housing 1701 having a surface 1703 to be directed at a treatment area. In order to retain an appropriate shape for airflow control and withstand stresses and forces associated with operation, the housing 1701, in some embodiments, can be constructed of any suitable material such as metals, plastics, transparent plastics, glass, polycarbonates, polymers, sapphire, any other suitable material, or combinations thereof. To the extent that it is desirable to permit the EMR to be transmitted through the housing 1701 to be directed to the treatment area, it may be advantageous to form at least a portion of the housing 1701, in particular at least a portion of the surface 1703, from an optically transparent material. In some embodiments, the entire housing 1701 can be optically transparent. As shown in FIG. 17, in some embodiments, the housing 1701 may not be optically transparent while the surface 1703 is transparent. However, in general, portions of the surface 1703 proximate to or coincident with the EMR beam should generally be optically transparent so as not to interfere with transmission of the EMR.

To facilitate transmission of the EMR beam therethrough, the housing 1701 can also include an EMR port 1707 for engagement with the fiber output 209 to direct the EMR beam through the housing 1701, including the surface 1703, and onto the treatment area. In accordance with various embodiments, the EMR port 1707 can include any fitting capable of engaging the fiber output 209, such as, for example, a Luer slip, a Luer lock, a fitting, a fiber coupler, or any other suitable fitting. More generally, the EMR port 1707 can include any configuration suitable for directing an EMR beam generated by the fiber output 209 through the housing and toward the treatment area.

In some embodiments, the device 1700 can include beam shaping optics (not shown) for producing a particular beam shape. For example, as shown in FIG. 17, the beam shape can be an expanding square beam. However, although the EMR is shown in FIG. 17 as being an expanding square beam, it will be apparent in view of this disclosure that any other beam shape can be used in accordance with various embodiments, including, for example, expanding, converging, straight, homogenized, collimated, circular, square, rectangular, pentagonal, hexagonal, oval, any other suitable shape, or combinations thereof.

The device 1700, as shown in FIG. 17, can also serve as an air cooling apparatus for cooling the treatment area. To that end, the device 1700 can include one or more cold air ports 1709 for receiving airflow into the housing 1701. Each cold air port 1709 can be any suitable design, size, or shape for connecting to an airflow source, including, for example, an opening in the housing 1701, a tube in fluid communication with the housing, a luer lock connector, a luer slip connector, a fitting, any other suitable design, or combinations thereof. In some embodiments, the cold air port 1709 can be formed integrally with the housing 1701. In some embodiments, the cold air port 1709 can be a separate element attached to, fastened to, or otherwise in fluid communication with the housing 1701.

The airflow received into the housing 1701 via the cold air port 1709 can be directed through the surface 1703 toward the treatment area for direct air cooling of the treatment area. In particular, the surface 1703 can include a plurality of openings 1705 formed in the surface 1703 for directing airflow onto the treatment area. In some embodiments, the openings 1705 can be positioned to direct the airflow onto the treatment area at temperatures, flow rates, and exit flow velocities suitable to maintain the treatment area at a therapeutically acceptable temperature range while avoiding interference with the EMR being directed at the treatment area. To that end, openings 1705 coincident with or within close proximity to a portion of the surface 1703 through which the EMR is transmitted (EMR transmission region) can be formed from optically transparent material. To the extent that other openings 1705 are not aligned with the EMR transmission region, those openings may not need to be transparent.

In some embodiments, the plurality of openings 1705 can be arranged in a pattern that can provide substantially uniform cooling over at least the treatment area illuminated by the EMR. In some embodiments, the substantially uniform cooling can extend over an area larger than the treatment area. In such embodiments, pre and post cooling to the treatment area is permitted as the device 1700 is moved from one treatment area to another by the positioning apparatus 900, whether manually or by automated control by the controller 403 as programmed to deliver the appropriate energy to maintain the target temperature range for a procedure.

In order to promote a uniform flow and maintain a desired cooling rate, during use, the openings 1705 can be spaced apart from the target surface to maintain the substantially uniform cooling and to promote efficient jet impingement cooling. For example, in some embodiments, the spacing between the exit plane of the openings 1705 and the target surface can be maintained between zero (0) inches to more than an inch. In some embodiments, the spacing can be about 0.5 inches. More generally, any spacing between the openings 1705 and the target surface can be used so long as substantially uniform cooling can be provided to the treatment area to maintain a therapeutically acceptable temperature range.

The spacing and positioning of the device 1700 can generally be maintained by adjustment of the positioning apparatus 900 as described above with reference to FIG. 9. To facilitate positioning of the device 1700 by the positioning apparatus 900, the device 1700, in some embodiments, can include a device mount 1715 for operatively engaging the device 1700 with the positioning apparatus 900 (not shown in FIG. 17). For example, as shown in FIG. 17, the device mount 1715 can include a flange for removable engagement with the positioning apparatus 900. However, it will be apparent in view of this disclosure that any device mount 1715 capable of providing removable engagement with the positioning apparatus 900 can be used in accordance with various embodiments.

Although shown in FIG. 17 and described herein as including a device mount 1715 and as being mounted to the positioning apparatus 900, it will be apparent in view of this disclosure that, in some embodiments, the device 1700 may, in some embodiments, be used as a manual hand piece. In such embodiments, the device 1700 may not include a device mount 1715 and instead can be coupled to the housing 100 only by the fiber output 209 at the EMR port and/or a cooling air supply at the cold air port 1709 for permitting manual operation and positioning of the device 1700.

In particular, the spacing can be maintained by providing program instructions for the computing device 107 and the controller 403 for operating the positioning apparatus 900 responsive to real time feedback from one or more position sensors 1711 mounted to the housing 1701 and directed toward the treatment area. The position sensors 1711 can be configured to detect one or more of a distance between the device 1700 and the target area, an orientation of the device 1700 relative to the target area, and a position of the device 1700 on the target area. The position sensors 1711 can generally be any suitable sensor for providing non-contact detection of a position of the device 1700 relative to the target area. For example, as shown in FIG. 17, the position sensors 1711 can be infrared location sensors.

In order to aid in meeting procedure requirements, in some embodiments, the device 1700 can include one or more temperature sensors 1713 to provide real time monitoring of a temperature of the treatment area. In particular, as shown in FIG. 17, the temperature sensors 1713 can include one or more non-contact pyrometers to provide non-contact temperature monitoring of the treatment area. In some embodiments, the temperature sensors 1713 can be configured to provide real time temperature feedback to the computer 107 and/or the controller 403. The computer 107 and/or the controller 403 can then responsively adjust one or more operating parameters of the system 10 to maintain the target area at a therapeutically acceptable temperature. For example, in some embodiments, responsive to the temperature feedback provided by the temperature sensors 1713, the controller 403 can at least one of instruct the positioning apparatus 900 to adjust a spacing between the treatment area and the device 1700, instruct the positioning apparatus 900 to adjust a scanning velocity of the emitted EMR beam relative to the target area, instruct the pump 507 to adjust a flow rate of the cooling air or gas, instruct the refrigeration unit 501 to adjust a coolant temperature, thereby adjusting a temperature of the cooling air or gas, instruct the laser sources 203 to adjust a power of the emitted EMR beam(s), shut off or activate one or more of the laser sources 203, instruct the device 1700 to adjust beam shaping optics to alter a beam shape of the emitted EMR beam, or combinations thereof.

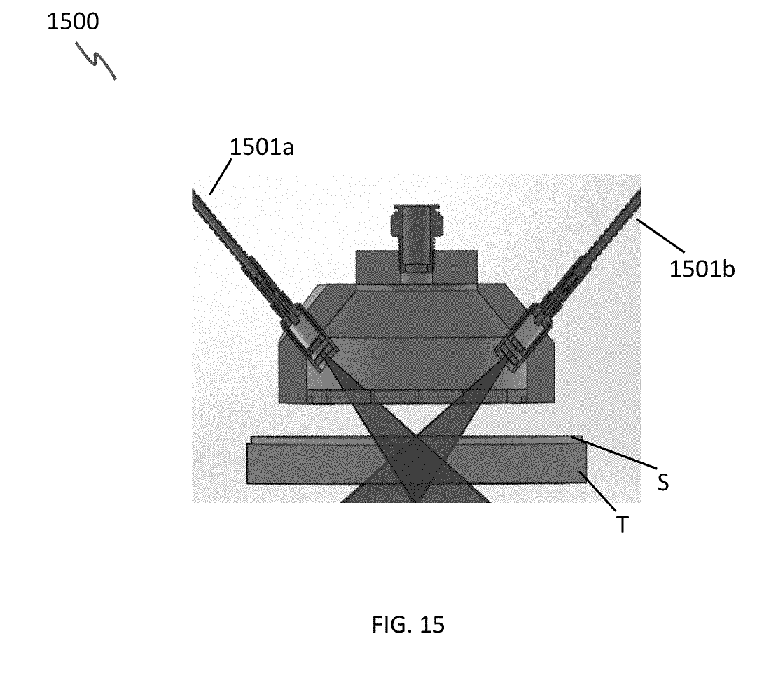

Referring now to FIG. 15, an device 1500 is illustrated wherein the common output cable 209 is split by a beam splitter (not shown) to provide two or more output cables 1501a, 1501b for emitting two or more beams, each delivering only a portion of the total EMR power transmitted by the common output cable 209. Alternatively, in some embodiments, rather than splitting a common output cable 209, the two or more output cables 1501a, 1501b can each be separate, unsplit output cables directly connected to a single laser source 203 and/or the combiner 207. In such embodiments, the array 200 can include a corresponding number of laser sources 203 each having a same wavelength to deliver beams having the same wavelength via each of the emitter cables 1501a, 1501b. Advantageously, such embodiments can permit the use of smaller, lower power, less expensive laser sources 203 because each emitter cable 1501a, 1501b is only required to deliver a portion of the total EMR power used for treatment of the treatment area.

The device 1500 is configured to direct the beams emitted from the output cables 1501a, 1501b at an angle such that the beams impinge separately on a surface to be illuminated S and overlap beneath the surface S in a subsurface tissue to be treated T. Such embodiments can generally provide a lower power density at the point of impingement on the surface S and a higher power density in the overlap region in the tissue T. In particular, power density in the overlap region will scale proportionally with the number of EMR output cables 1501a, 1501b, the power of each EMR beam, and the beam size of each beam in the overlap region. Accordingly, it will be apparent in view of this disclosure that any number of output cables producing any number of EMR beams can be used in accordance with various embodiments as desired to provide a desired power density at the surface S and in the overlap region of the tissue T. For example, in some embodiments, four beams can be provided wherein two pair of opposing beams can be configured in a square arrangement to emit beams at the slant angle to project a rectangular pattern onto the surface S and into the tissue T. In some embodiments, to overlap two more EMR beams from opposing but orthogonal locations, each beam footprint can be rectangular to create a similar projected beam foot print on the treatment plane. More generally, the beam shape of each EMR beam, in some embodiments, can, for example, be diverging, collimated, converging circular, square, rectangular, any other suitable shape, or combinations thereof.

Such a configuration is advantageous because, during, for example, a procedure for hyperthermia of adipose tissue to create apoptosis, the objective is to reach temperatures in the fat (adipose) tissue roughly from 42 to 47° C. During this process where the fat tissue is positioned beneath the skin and epidermis by approximately 2.8 mm, the skin, including the active nerve endings therein, can reach temperatures that feel warm or even hot to the patient. Although cold air or cryogenic cooling is typically provided, higher EMR power densities may nevertheless raise skin temperature to an uncomfortable temperature. In such cases, splitting the EMR power into two or more beams impinging separately on the surface of the skin can reduce local skin heating. On the other hand, the sum power of all overlapping beams is concentrated where the EMR beams overlap. Because maximum power is achieved in the overlap region, higher temperatures can be achieved in the overlap region for more efficient apoptosis. Conversely, the lower power density on the skin, epidermis, and dermis will result in lower temperatures in those regions. In some embodiments, such lower power density can reduce skin cooling requirements for maintaining patient comfort and safety during the treatment.

Additionally, by setting or adjusting beam impingement angle of the beams emitted by the output cables 1501a, 1501b, a depth of tissue treatment can be controlled. In particular, by decreasing the angle of the multiple beams relative to vertical, the overlap region can be formed deeper into the tissue and/or extend deeper into the tissue. Advantageously, by overlapping the beams deeper in the tissue T, more tissue T can be treated during a procedure. Additionally, deeper treatment areas can target different, deeper tissues T than single beam systems or systems having a shallow overlap region. Thus, particular selection or adjustment of slant incident angles, including, for example, from about three (3) degrees to about 75 degrees, can provide high EMR power targeted at a desired depth in the desired tissue T without overheating the impingement surface S.

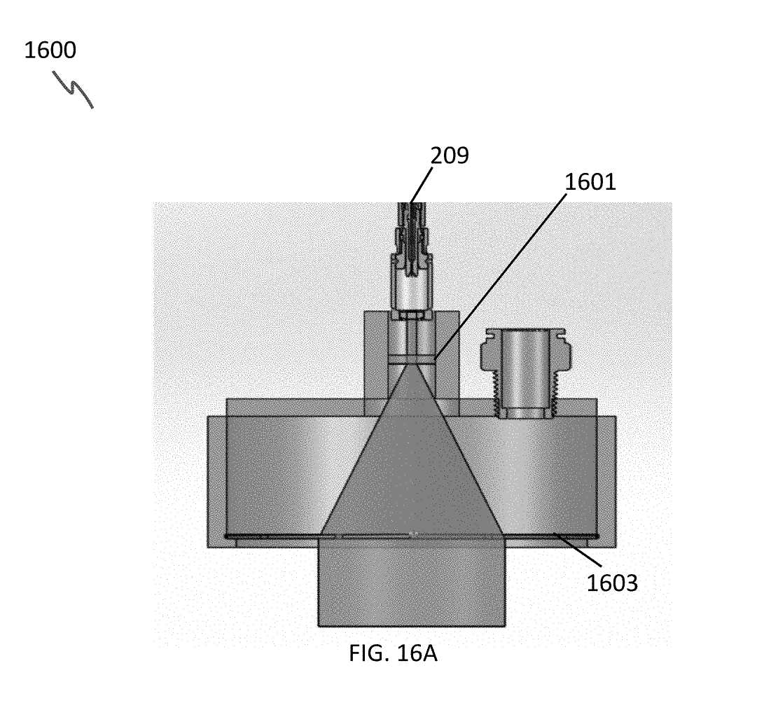

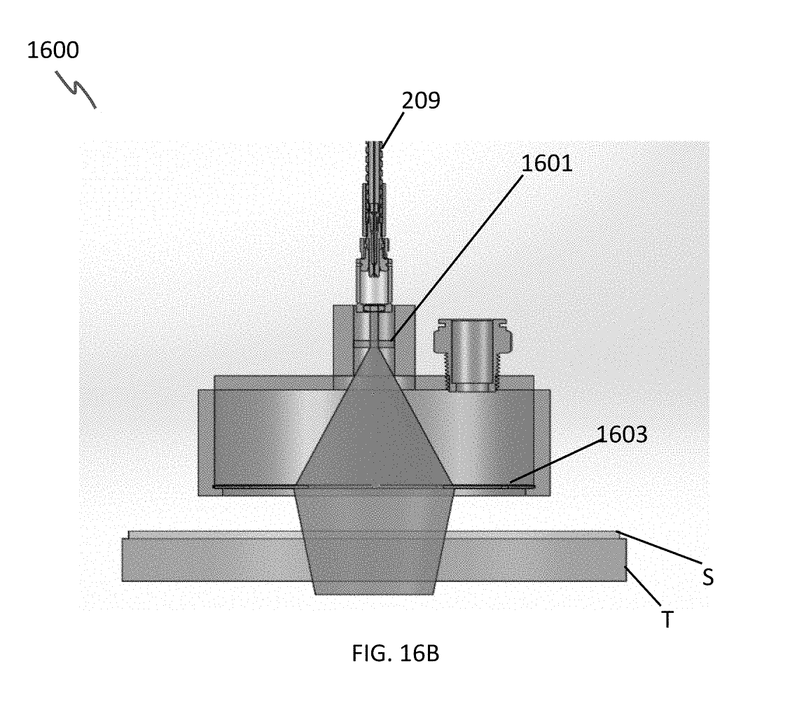

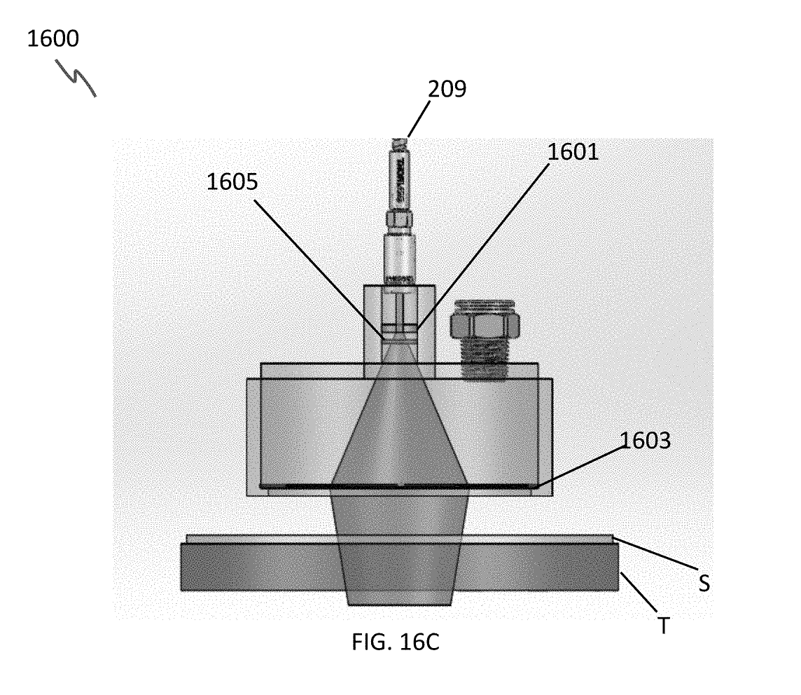

Referring now to FIG. 16A, in some embodiments, an device 1600 can include one or more optical elements for expanding, homogenizing, and refocusing EMR energy to aid treatment. In particular, a straight beam directed at a surface S to be illuminated can concentrate the EMR power in a small treatment area, making temperature management difficult and requiring additional movement and time to treat a target tissue T. Thus, in some embodiments, the device 1600 can include a beam expander 1601 to expand a size of a beam emitted by the common output cable 209. In particular, the beam expander 1601 of FIG. 16 is shown as a diffractive optical element (DOE) beam expander 1601. However, it will be apparent in view of this disclosure that any beam homogenizer, beam expander, or combination thereof can be used in accordance with various embodiments.

For applications where the target tissue T is beneath a surface S to be illuminated (e.g., where apoptosis of adipose tissue is desired), a beam expander 1601 alone would cause the beam power to be most diffuse in the target tissue T. Such a configuration makes heat management of the illuminated skin more difficult because the skin surface S is exposed to more concentrated beam power and thus heats up more quickly than the target tissue T. Therefore, in some embodiments, the device 1600 can also include a Fresnel objective lens 1603 for refocusing the expanded beam. As shown in FIG. 16B, in some embodiments, adjusting a spacing between the DOE beam expander 1601 and the Fresnel objective lens 1603 can adjust the focus. Thus, in some embodiments, the beam can be adjusted to be narrower (more concentrated) in the target tissue T and more diffuse at the surface S such that the skin surface S heats more slowly than the target tissue T. Referring now to FIG. 16C, in some embodiments, a negative Fresnel lens 1605 can be positioned between the beam expander 1601 and the Fresnel lens 1603 to permit additional beam shaping.

Referring again to FIG. 1, the system 10, in some embodiments, can include one or more sensors 1000 for monitoring operational conditions such as temperature of the treatment area. In some embodiments, the sensors 1000 can be configured to provide real time feedback to the computing device 107 so that the computing device 107 can, if desired, provide instructions to one or more components of the system 10 to alter one or more operational properties of the system 10 in response to the feedback. For example, in some embodiments, the positioning apparatus 900 can be instructed to scan the target area faster or slower to decrease or increase dwell time, move the device 950 closer to or further away from the target surface, reposition the device 950, temporarily suspend treatment, terminate treatment, increase or decrease cooling flow through a patient cooling system.

To the extent that patient temperature data is required, in some embodiments, to maintain a therapeutically acceptable temperature range, a subcutaneous temperature prediction sensor 1000 can be provided. By way of background, various tools and methods in the prior art have tried to non-invasively measure core or fat temperatures in the human body. Some rely on blackbody radiation signals in the microwave region. Others employ temperature sensors, in combination with estimated skin and tissue thermal conductivity, to predict the core temperature. These types of devices are too large, complicated or expensive to be applied to normal aesthetic treatment settings. Some devices have attached heated sensors to the skin with temperature sensors to predict core temperatures. Other approaches have monitored the skin surface temperature and the energy input.

Invasive temperature measurements are possible but not preferred due to the associated risks, and desire for a fully non-invasive hyperthermia treatment. Elaborate instruments such as MRI (Magnetic Resonance Imaging) or advance ultrasonic devices are capable of these measurements, but involve expensive and large devices which are also not readily used during many treatments.

A non-invasive sensor 1000 for measuring a core body fat temperature of a patient, the sensor 1000 can include a temperature sensor 1001 for measuring skin surface temperature and a heat flux sensor 1003 for measuring heat flow into or out of the treatment site. In some embodiments, the temperature sensor 1001 can include, for example, a thermocouple or a non-contact pyrometer. In some embodiments, the heat flux sensor 1003 can include, for example, a thermopile or a Seebeck effect sensor.

The sensor 1000 can then continuously monitor temperature and heat flux of the patient during treatment and feed that data back to the computing device 107 for processing. The temperature and heat flux data can be synthesized in an algorithm with user input data such as patient skin type, age, size, body fat percentage, etc. to estimate a temperature of the target subcutaneous fat. The computer system 107 can then adjust one or more operating parameters such as pulse length, EMR source activation, EMR source power, treatment duration, cooling airflow, scanning speed of the positioning apparatus, etc. to manage the temperature in response to the sensor 1000 feedback. Although shown as including both a temperature sensor 1001 and a heat flux sensor 1003, it will be apparent in view of this disclosure that, in some embodiments, the sensors 1000 may include only a temperature sensor 1001 or only a heat flux sensor 1003.

In some embodiments, the continuous temperature monitoring can begin with a numerical finite element simulation of fat region heating under EMR illumination to predict temperature over time and EMR source modulation. In particular, EMR source heating is applied in time dependent modulation and diminishes with depth of penetration. As the procedure progresses, skin temperature and skin heat flux are measured for the patient using the temperature sensor 1001 and the heat flux sensor 1003. Then, the temperature and heat flux data, the patient's unique data, and the finite element model are entered and combined in an overall algorithm to control the radiation input actively and maintain fat temperature in the effective range.

The measured parameters of a patient's skin temperature and skin heat flux in cooled regions can be measured several ways. Skin surface temperature can be made by a non-contact optical pyrometer recording in the radiated region, or a thermistor or thermocouple package. Temperature will be monitored before, during, and after EMR source irradiation. The rate of change of the skin temperature is monitored in the algorithm. The skin heat flux is derived in a non-contact method using the surface temperature measurement in combination with actively monitored cooling flow rate. When the two measurements are included in a heat transfer algorithm, calculation of skin heat flux is possible. Alternatively, a surface heat flux sensor can provide heat flux data.

Patient data used in this algorithm includes skin type and pigment, gender, age, size, weight, body mass index, and possible pretreatment history and skin distinctions. When available, more detailed tissue data can be entered. Tissue profiling collected from MRI's or ultrasonic devices can also provide accurate parameters to be incorporated into the tissue model. Other technologies such as non-invasive body core temperature measurement instruments that use black body radiation in the microwave region can be applied. Patient factors such as skin pigment characterization are important to estimate the anticipated EMR transmission and absorption values.

The algorithm is used to control the EMR energy delivered to a treatment area, known as fluence, in watts per square centimeter, as well as the exposure durations. The hyperthermia adipose reduction is normally done with on-off modulations and possible movement of beam location, which returns to reheat a region to maintain effective temperature range. The skin cooling is expected to be controlled based on skin surface temperature feedback for comfort level (e.g. 30° C.) and maximum safe temperature (e.g. 40° C.). The entire treatment period can last from several minutes to more than 30 minutes.

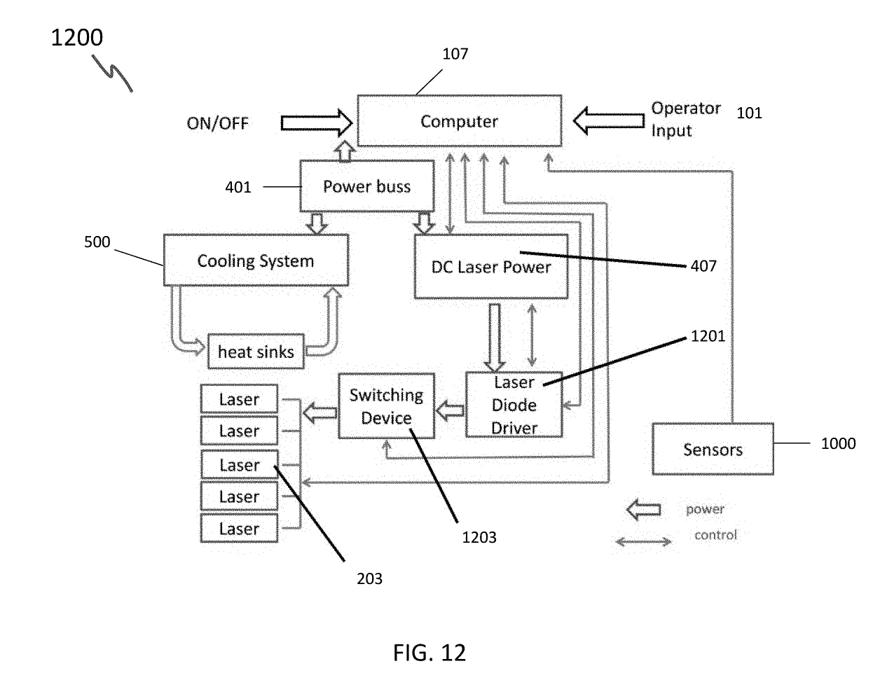

Referring now to FIG. 12, a schematic of a system 1200 for electronics and control of a multifunction aesthetic system having a single diode driver is provided. In particular, the high voltage ADC 411 can operate several laser sources 203 from a shared diode driver module. In this case, multiple laser sources 203 of the same voltage/current requirements are operated from a single diode driver. In some embodiments, the system 1200 is substantially similar to the system 400 of FIG. 4. However, the system 1200 of FIG. 12, includes a single diode driver 1201 and a switching device 1203 interposed between the diode driver 1201 and the laser sources 203 to permit the diode driver 1201 to selectively drive a desired one of the laser sources 203.

The diode driver 1201, in some embodiments, can be substantially similar to the diode drivers 409 discussed above in connection with FIG. 4. The switching device 1203, in some embodiments, can be configured to switch the driver 1201 between the diode load of each laser source 203 as required. In some embodiments, the switching device 1201 can include one or more high current mechanical relays, one or more solid state relays (SSR), or both.

The switching device 1203 can be placed on ‘high side’ of the diode driver and the relays can be selected one at a time to drive a particular laser source 203. The relays must be capable of handling the current driven to the selected laser source 203. The relays or SSRs can be used as a safety interlock (emergency power cut) for the laser sources 203 as well. However, in the configuration of FIG. 12, multiple laser sources 203 cannot be driven by selecting more than one relay at a time. Such a configuration would place the laser sources 203 in parallel with each other and the driver 1201. Even if the driver 1201 is capable of sufficient current, there is no passive or active load sharing between the two laser sources 203. Because one of the diodes will have a lower resistance, that device will ‘hog’ the current, over power, and burn out, leaving the second channel to do the same. Because such burnout can happen very quickly (seconds), the switching device 1203 must be configured to select only one diode at a time. Additionally, switching the diode channel must occur when the driver is off. In particular, diode laser sources 203 operate at a near short (about 3 milliohms for a diode bar). Therefore, if the output of an active driver is switched from an open load to a diode load, a large overcurrent spike will occur, likely damaging or destroying the diode.

When deciding between SSR and mechanical relays, SSRs tend to be faster, more reliable, and don't typically require electrically isolated control lines. However, isolated input SSRs allow the use of a single driver for several diodes with less concern for ground loop issues. In addition, in the event of a failure, an isolated SSR input will provide a buffer for the sensitive control circuitry.

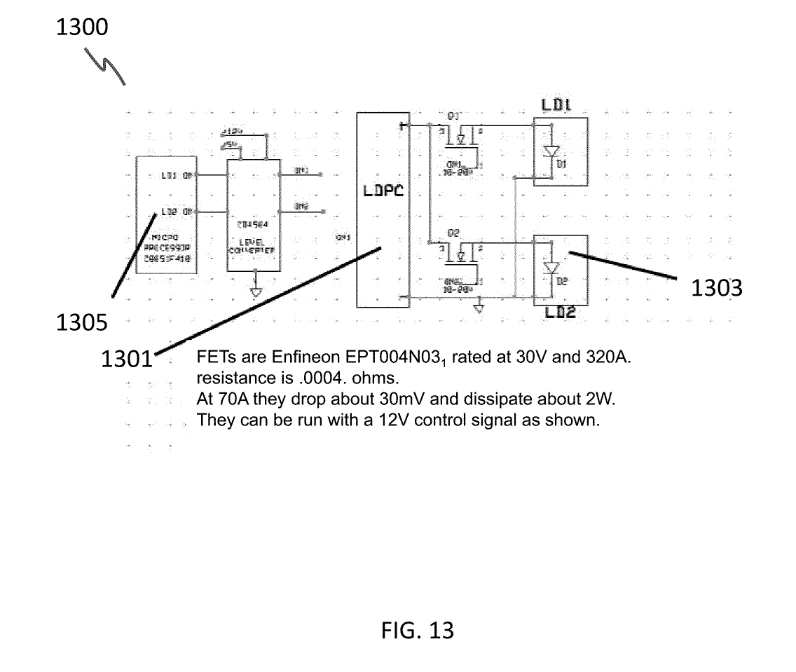

Referring now to FIG. 13, in some embodiments, the switching device can employ a single Diode Driver Printed Circuit (DPC) 1301 to power multiple EMR sources 1303 is shown. The high current capacity FET's can be used as switching devices to activate and power the selected EMR source. This diagram shows only two drivers (LD1 and LD2), but the same concept can be applied to drive multiple EMR sources. The control input to the switching FET's is routed from the processor 1305. This design approach eliminates the need for switching relays with the command signal driving only the selected driver and therefore activating that EMR source.

### Example Embodiment

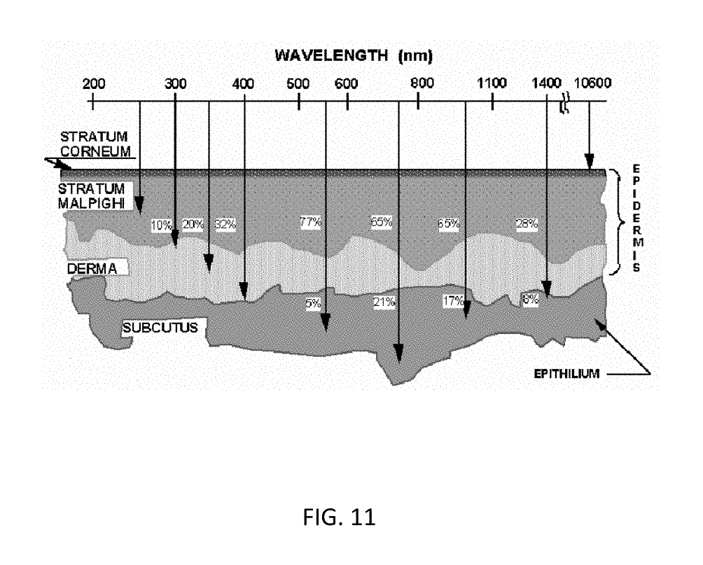

In one embodiment, it may be desirable to perform subcutaneous fat reduction and skin tightening simultaneously. However, as shown in the human tissue profile of FIG. 11, different EMR wavelengths have different expected penetration depths. In particular, FIG. 11 illustrates, by percentage, for each wavelength, the percentage of EMR energy penetrating to various depths. More generally the fat is typically more than 5 mm from the skin's surface. Thus, for example, a wavelength of about 1064 nm (e.g., 400 nm to 3000 nm or 900 nm to 1100 nm) can be selected for hyperthermia of fat tissue because it exhibits good transmission through the skin, epidermis, and dermis and deposits energy within the fat cells. On the other hand, skin tightening generally requires other wavelengths that exhibit higher absorption in the epidermis and dermis, where the collagen resides. Thus, for example, a wavelength of about 400 nm to about 3000 nm or about 1300 nm to about 1400 nm. These EMR beam wavelengths deposit more energy to the collagen, creating necrosis and eventually skin tightening from new collagen regrowth.

In such an embodiment, the controller 403 of the power and control electronics 400 of the multifunction aesthetic system 10 described herein can activate a first driver 409/laser source 203 pair to produce an EMR beam having a wavelength suitable for subcutaneous fat reduction while simultaneously activating a second driver 409/laser source 203 pair to produce an EMR beam having a wavelength suitable for skin tightening. In some embodiments, such a procedure can also be used in conjunction with other fat reduction techniques such as procedures using RF (radio frequency), MW (microwave), ultrasonic, or cryo (cold therapy) fat reduction methods.

In further example, in some embodiments, the methods described above can be used to activate driver 409/laser source 203 pairs for emitting wavelengths suitable for performing any other procedure or combination of procedures including, for example, but not limited to, fat reduction, body skin tightening, facial skin tightening, skin resurfacing, skin remodeling, vein reduction or removal, facial pigment removal or reduction, hair removal, acne treatment, scar reduction and removal, psoriasis treatment, stretch mark removal, nail fungus treatment, leukoderma treatment, tattoo removal, or combinations thereof as discussed above.

### Systems and Methods for Characterizing Skin Type

Embodiments of the present disclosure generally provide systems and methods for characterizing skin type using an optical sensor and multispectral illumination sources. In various embodiments, the skin may be illuminated by light of various wavelengths generated by the multispectral illumination sources, and the optical sensor may receive light reflected from the skin as illuminated. The light received by the optical sensor, in various embodiments, may be processed to identify characteristics indicative of the skin type(s) found in the illuminated area. These characteristics, in various embodiments, may be processed in accordance with one or more algorithms to automatically characterize the skin type(s). As configured, systems and methods of the present disclosure, in various embodiments, provide for real-time or near real-time characterization of skin type(s), which can in turn be used to improve the efficiency, efficacy, and safety of providing a wide variety of skin treatments. For example, the knowledge of skin type is useful for aesthetic and dermatological procedures including hair and tattoo removal, skin tightening, skin resurfacing, as well as for determining suitable intensity and dosage levels in procedures involving the application of intense light.

As used in the present disclosure, the term “skin type” and derivatives thereof refers broadly to a characterization of one or more properties of the skin that may contribute to the skin's sensitivity and reaction to one or more wavelengths of light, acids, bases, chemicals, or any combination thereof. One traditional approach for characterizing skin type is known as the Fitzpatrick Scale. This approach is often performed visually by a dermatologist, clinician, or the like, and involves visual comparisons of skin color to a standard color chart, along with an interrogation of the subject to questions regarding sensitivity to burning during exposure to direct sunlight. Based on the color chart comparison and subject feedback, the subject's skin type is then classified by the dermatologist into one of six types, ranging from Type I (pale while, freckled skin that always burns and never tans) to Type VI (deeply pigmented dark brown skin that never burns and never tans). In various embodiments, skin type may be characterized using classifications similar to those of the Fitzpatrick Scale. It should be understood; however, that the present systems and methods can provide significantly more detailed information concerning skin properties than that available from simple visual inspection by a dermatologist, and thus are able to classify skin type in the context of properties relative to a given treatment in far more precise and useful terms, as later described in more detail.

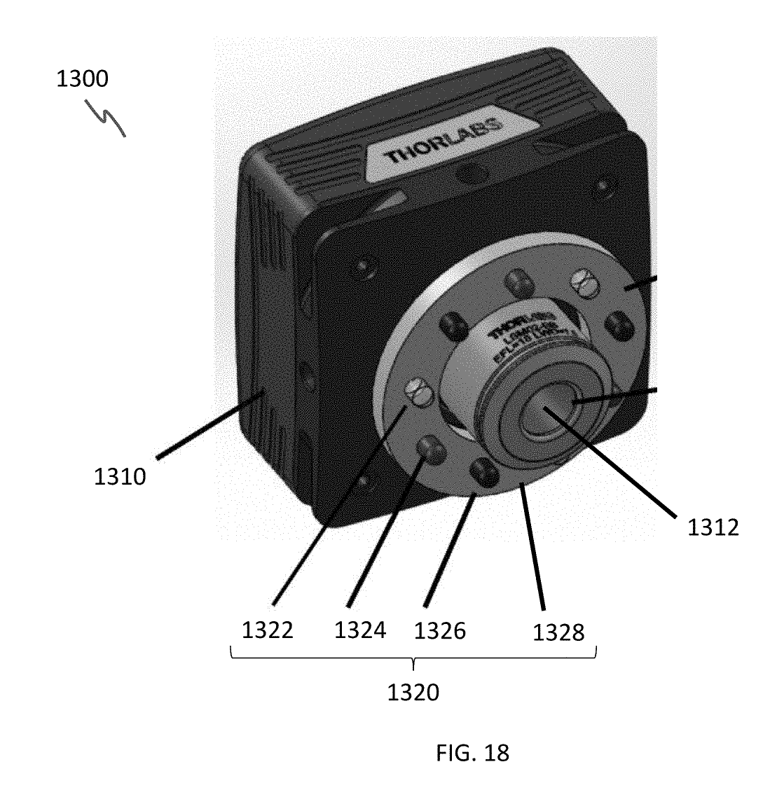

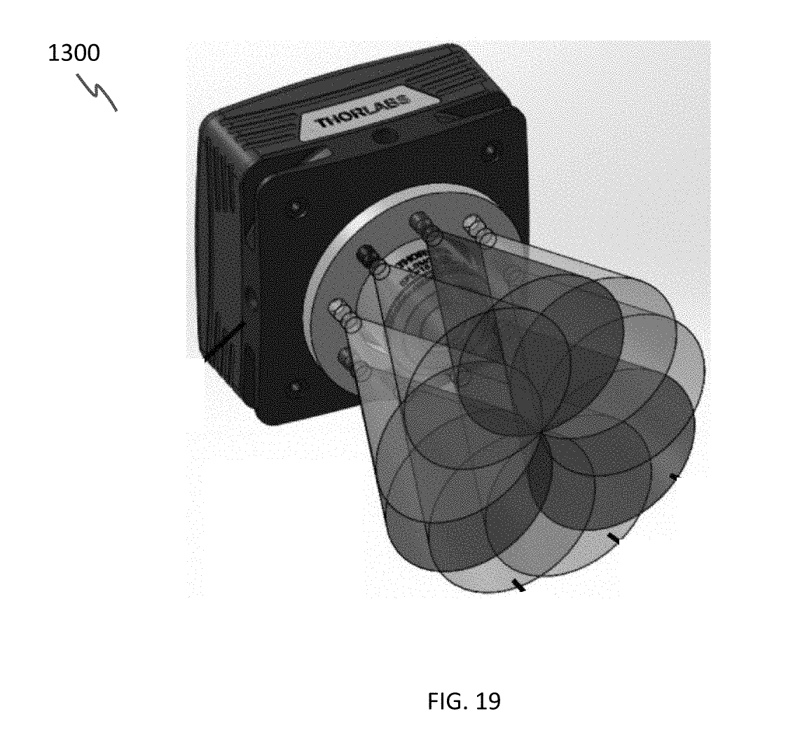

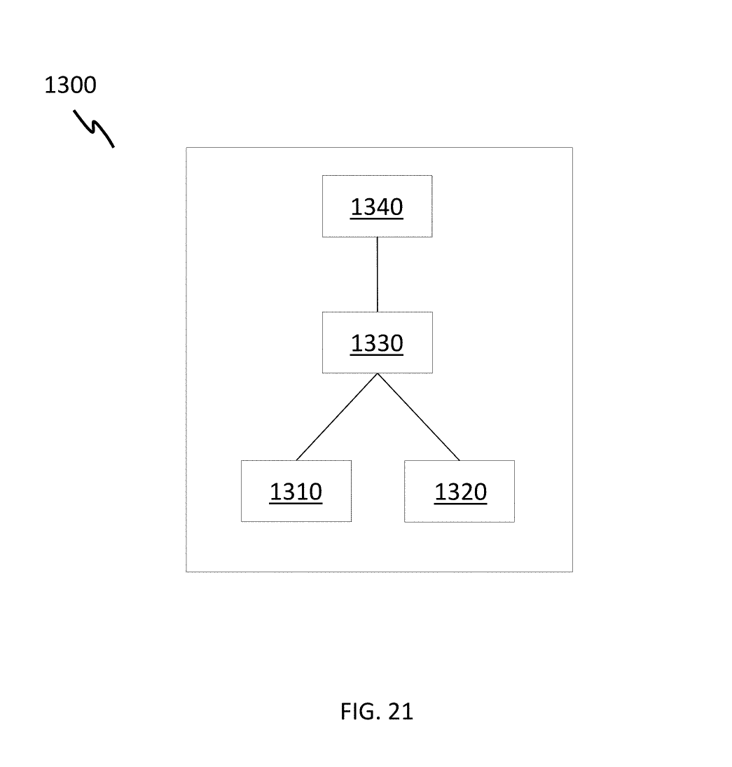

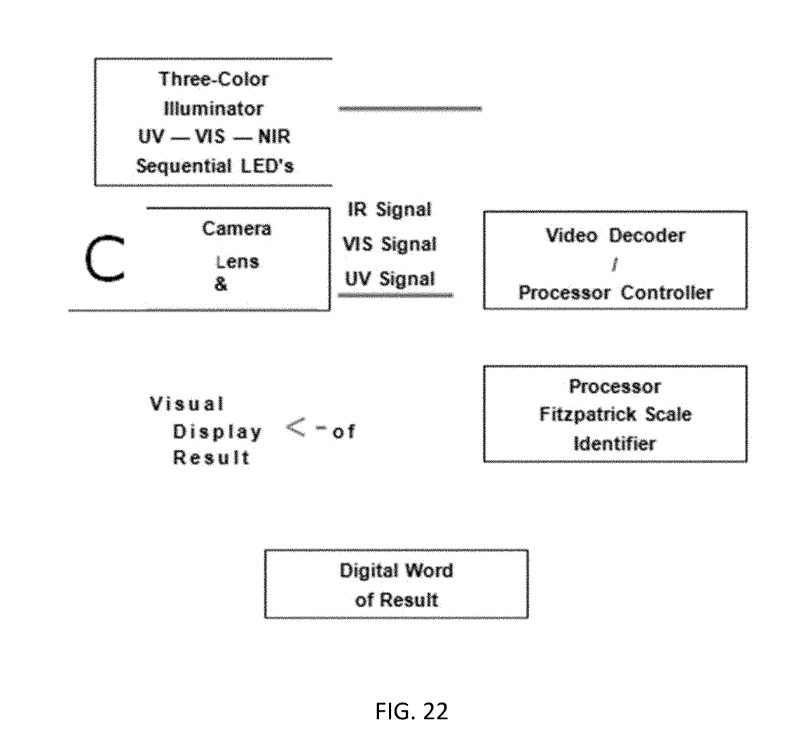

FIGS. 18 and 19 depict a representative system 1300 for automatically characterizing skin type. As shown in FIGS. 18 and 19, in combination with the corresponding block diagrams of system 1300 depicted in FIGS. 21 and 22, system 1300, in various embodiments, may generally include an optical sensor 1310, a multispectral illumination source 1320, a processor 1330, and a memory 1340.

Generally speaking, the skin may be sequentially illuminated by light of various wavelengths generated by the multispectral illumination sources 1320, and optical sensor 1310 may receive light reflected from the skin as illuminated by each such wavelength. Optical sensor 1310, in various embodiments, may convert the received light into a video or similar signal. In various embodiments, processor 1330 associated with at least optical sensor 1310 may process the video or similar signal to measure one or more properties of the light received by optical sensor 1310, such as quantitative measurements of the spectral reflectivity of the skin. Processor 1330, in various embodiments, may then apply methodologies in accordance with instructions stored in memory 1340 of system 1300 to automatically characterize skin type based at least in part on the measured properties, as later described in more detail.

### Optical Sensor 1310

Optical sensor 1310 of system 1300, in various embodiments, may include any optical sensor(s) suitable for converting light, or a change in the light, reflected from the skin area being illuminated by multispectral illumination source 1320 into an electronic signal. Representative examples of suitable optical sensors may include an image sensor (e.g., CCD, CMOS), a digital camera, and the like.

In various embodiments, optical sensor 1310 may be configured to convert reflected light corresponding with the wavelengths of light generated by multispectral illumination source 1320. For example, many optical sensors, such as a digital camera, are capable of detecting light with wavelengths ranging from 200 nm to 1500 nm when conventional blocking filters are removed. It should be recognized that light emitted from multispectral illumination source 1320 may, in some cases, undergo a change in properties such as wavelength, amplitude, frequency, and the like upon being reflected off of the skin, and thus, in various embodiments, optical sensor 1310 may be configured to receive and convert light having such altered properties into an electrical signal.

As shown in FIGS. 18 and 19, in an embodiment, optical sensor 1310 may comprise a lens 1312 for focusing in on a particular area of the patient's skin. Lens 1312 may be used alone or in combination with positioning optical sensor 1310 closer or farther away from a target area of interest for a similar purpose.

The electrical signal, in various embodiments, may include any suitable signal and signal format for communicating information concerning the light received by optical sensor 1310 for processing, as later described in more detail. In a representative embodiment, the electrical signal may include a video signal, such as composite video.

### Multispectral Illumination Source 1320

Multispectral illumination source 1320 of system 1300, in various embodiments, may be configured to generate light of various wavelengths for illuminating the patient's skin. Representative wavelengths for use in characterizing the patient's skin type(s) may span a wide spectral range including wavelengths ranging from ultraviolet (UV) spectrum to near infrared (NIR). Illumination source 1320 may include any device suitable for generating light for illumination of the patient's skin, such as light emitting diodes (LEDs), laser diodes, incandescent bulbs, fluorescent lamps, and the like In the representative embodiment shown in FIGS. 18 and 19, multispectral illumination source 1320 may include one or more LED 1322, 1324, 1326 for generating light in the visible, NIR, and UV spectrums, respectively. Here, illumination source 1320 includes three of each such LED positioned about lens 1312 of optical sensor 1310 to cover the field of view of optical sensor 1310.

As shown in FIGS. 18 and 19, in various embodiments, the one more illumination sources (e.g., 1322, 1324, 1326) may be positioned near or on optical sensor 1310 such that the light and the field of view of optical sensor 1310 are generally aligned. This may make operation of system 1300 simpler as an operator need not separately position and orient optical sensor 1310 and multispectral illumination source 1320 when analyzing a particular area of the skin. In various embodiments, the one or more illumination sources (e.g., 1322, 1324, 1326) may be provided on or as part of a common platform, such as disk-shaped adapter mount 1328, thereby allowing multispectral illumination source 1320 to be coupled as a unit to optical sensor 10. As shown, in an embodiment, multispectral illumination source 1320 may be positioned about lens 1312. Of course, in various embodiments, multispectral illumination source 1320 and/or any of its constituent components (e.g., sources 1322, 1324, 1326) may be located separate from optical sensor 1310 and in any arrangement suitable for illuminating the skin area to be examined.

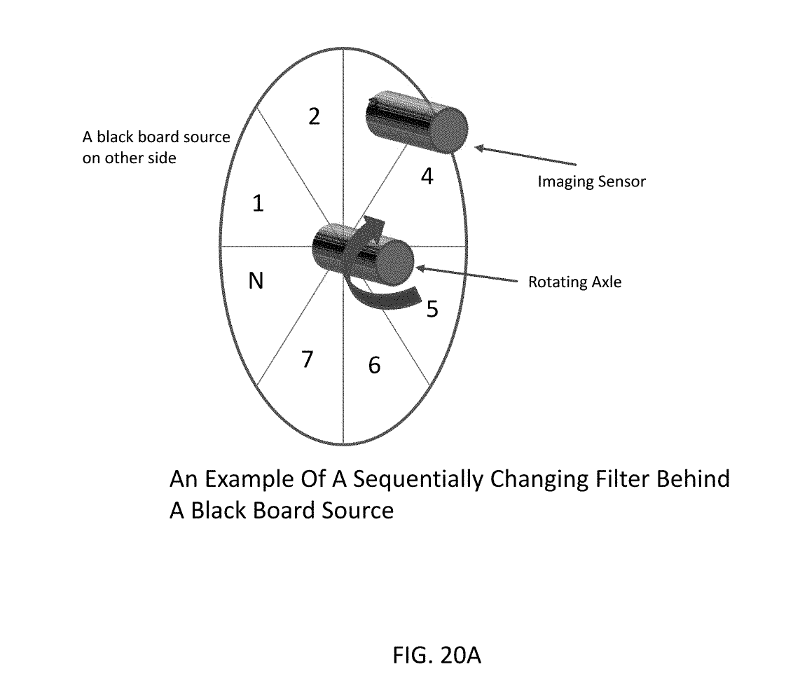

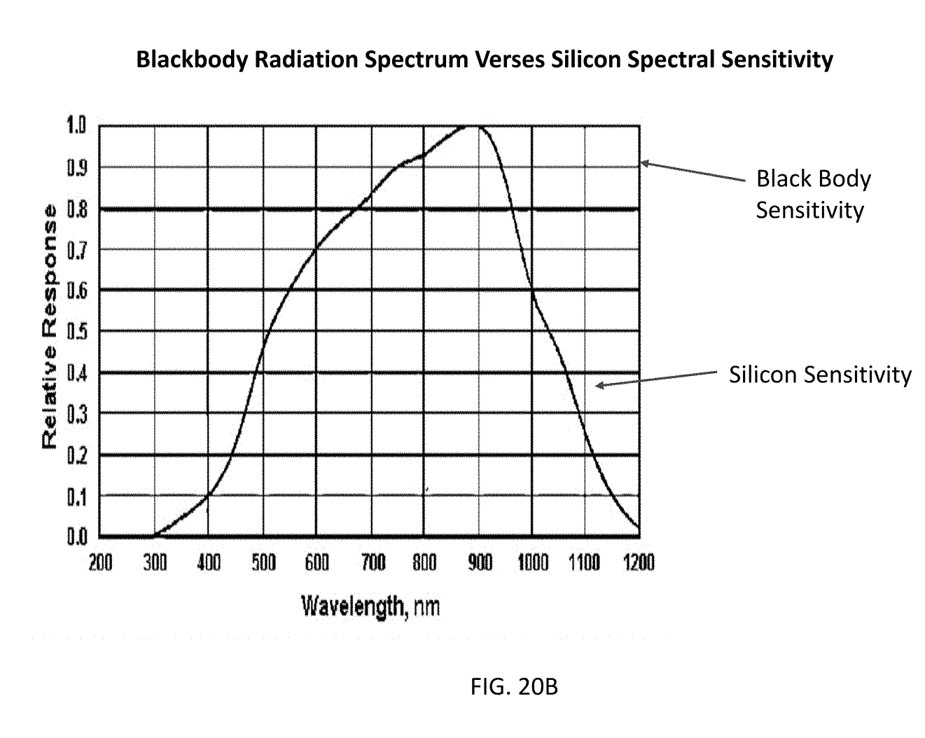

Referring now to FIGS. 20A and 20B, in various embodiments, illumination source 1320 may additionally or alternatively include a single blackbody radiation source configured to emit multiple wavelengths. In a representative example, such a source may include a high intensity incandescent light bulb having a continuous spectrum of light as shown in FIG. 20A. A range of spectral filters, in an embodiment, could be placed in front of image sensor 1310 and sequentially changed such that only that light within a desired spectrum reaches sensor 1310. In one such embodiment, image sensor 1310 may include a full range silicon CCD image camera. As shown in FIG. 20A, in a representative embodiment, a multispectral filter could take the form of a spinning wheel with multiple filters distributed about the azimuth.

### Processor 1330

Processor 1330 of system 1300, in various embodiments, may include any processor suitable for processing the electrical signal output from optical sensor 1310 to identify one or more relevant properties of the reflected light received by optical sensor 1310 for use in characterizing the skin type of the area being examined. In various embodiments, processor 1330 may be configured to execute instructions stored in memory 1340 for these purposes.

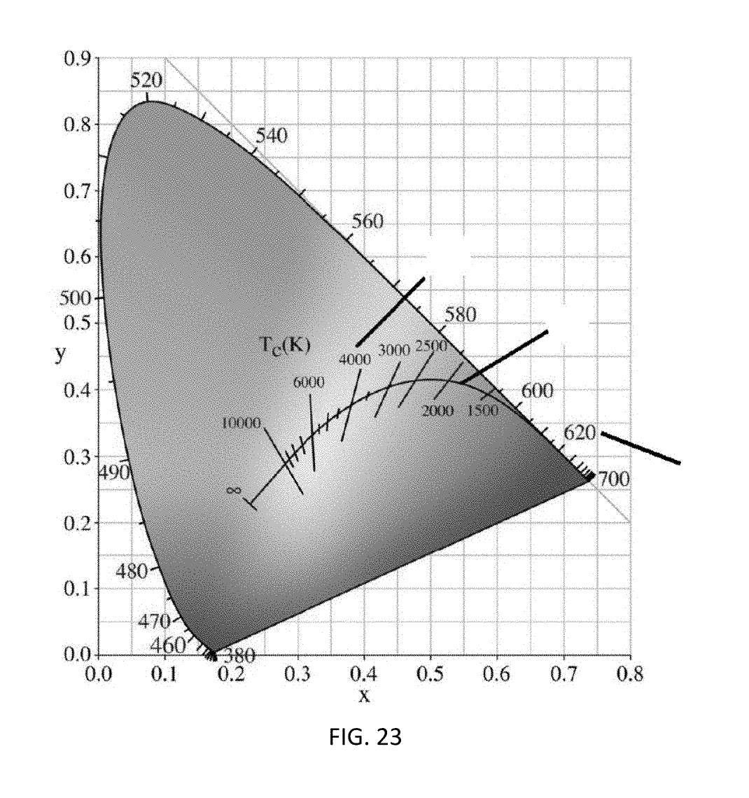

To that end, processor 1330, in various embodiments, may be configured to process the reflected light received by optical sensor 1310 to identify one or more relevant properties of the reflected light for use in characterizing the corresponding skin type(s) of the area being examined. In an embodiment, one such property may include the intensity of the reflected light for a given wavelength emitted by illumination source 1320. The intensity of the reflected light can, in turn, be correlated through one or more algorithms, either alone or in combination with other relevant light parameters, to characterize skin type. Additionally or alternatively, another useful property that could be processed by processor 1330 is skin coloration (and variations therein across the area being examined). In an embodiment, processor 1330 may process light reflected off of the skin to characterize color using any suitable scale, such as the CIE color scale illustrated in FIG. 23, which depicts unique coordinates for defining any color measured by processor 1330. It should be recognized that processor 1330, in various embodiments, may be configured to process the reflected light for one or more additional parameters indicative of spectral responsivity of skin as illuminated by illumination source 132, such as those indicative of concentrations of various chemical chromophores as later described in more detail. Each specific chromophore is known to play a role in overall photo-response of the skin to illumination by light sources used in a treatment modality, as further described for example, in “The Optics of the Human Skin” by Anderson, which is hereby incorporated by reference in its entirety for all purposes. These parameters may be used to identify and characterize relevant constituents in the skin that may be useful for characterizing skin type overall.

In various embodiments, processor 1330 may be configured to perform a calibration step prior to or after taking measurements for use in characterizing skin type. For example, in a representative embodiment, processor 1330 may be configured to compare measured parameters, such as the intensity and color of the reflected light, against corresponding properties of corresponding light reflected off of a standard white target.

The unique color and quality of skin type, which can vary with ethnicity and skin condition (e.g., light or tanned) of the subject, may be determined by a number of constituent components within the skin. Representative components may include, without limitation, melanin, Hb, HbO2, and bilirubin, amongst more complex proteins within the skin. Thus, evaluating a single or even a few color responses may not provide an in depth view of the real makeup of the skin and its photopic response. Accordingly, system 1300, as configured with multiple illumination wavelengths and image processing techniques for evaluating multiple parameters of the reflected light, may provide very detailed and robust information for characterizing skin type with fidelity far outpacing current approaches in the art.

Processor 1330 of system 1300, in various embodiments, may be further configured for automatically characterizing the skin type of the area being examined based at least in part on the above-referenced properties identified by processor 1330. In various embodiments, processor 1330 may be configured to execute instructions stored in memory 1340 for these purposes.

In particular, in various embodiments, processor 1330 may be configured to process one or more algorithms suitable for characterizing skin type using at least one of the one or more properties as inputs. The proper determination of the Fitzpatrick skin type is more complex than simply identifying the skin tone or color. The photo-responsive constituents of the skin, mainly melanin, have differing characteristics of reflectivity and absorption. These constituent molecules determine the response of the skin to various wavelengths, intensities and duration of treatment light sources. The skin responsivity, classified by the Fitzpatrick value, can best be characterized by illuminating the skin with light of several different wavelengths and then recording the response of the camera to the reflected component of the incident light.

In various embodiments, processor 1330 may be configured to generate a data file for each illuminating wavelength, recording the measured parameters of the reflected light such as intensity and color. The information contained in the data file may, in turn, be used as inputs in one or more algorithms for computing a full spectral curve. Taken together, the data may provide a spectral reflectivity curve which can be subsequently broken down mathematically to determine the levels of important constituents of the skin and their concentrations. This information, in turn, may provide a basis for characterizing skin type. In an embodiment, the full spectral curve may be evaluated for certain peaks, dips, and/or other features indicative of certain skin components. A magnitude of the feature, in an embodiment, may then be related to the concentration of that particular constituent(s) in the skin. Certain shapes in the spectral curve for reflected UV light, for example, may relate to susceptibility of the skin to burning. In some embodiments, this may be treated as a subjective quality for use in characterizing skin type, while in other embodiments, this susceptibility may be quantified given its magnitude and any other defining properties on the spectral curve and used to provide additional fidelity in skin type characterization.

Processor 1330, in various embodiments, may be further configured to identify any variations in skin type within the given area being examined by system 1300 by applying the above-referenced methodologies to multiple portions of the area illuminated by multispectral illumination source 1320 within the field of view of optical sensor 1310. In various embodiments, processor 1330 may be configured to execute instructions stored in memory 1340 for these purposes.

In some embodiments, processor 1330 may additionally associate the skin type characterization associated with each such portion with information indicative of the respective locations of each such portion within the area being examined. As configured, a map or other visual aid may be generated from the skin type and associated location data for visually presenting the variations in skin type as distributed across the area being examined. Such visual aids may be used by a dermatologist, clinician, etc. in assessing treatment options and tailoring a given treatment option to best complement the varied skin types found across the various portions of the area examined. For example, color coding could be used to generate a “heat map” type map, using hot shades (e.g., reds, oranges, yellows) to depict locations with more sensitive skin types and cool shades (e.g., violets, blues, greens) to depict locations with less sensitive skin types. Alternatively, a single color could be used and its intensity varied to indicate differences in skin type within the area being examined. In an embodiment, color coding could be presented with some transparency, perhaps with the ability to adjust said transparency, such that a person viewing the map may simultaneously or selectably see the color coding and the skin image itself. In an embodiment, the map may be interactive, allowing the clinician to, for example, select a particular area (e.g., using a mouse or touch screen) to display more detailed information concerning skin type for that particular area. It should be appreciated that such maps or visual aids may be generated from the measurements provided by system 1300 and corresponding location information provided by apparatus 900 using techniques known in the art.

In an embodiment, processor 1330 may additionally or alternatively associated the underlying properties used to characterize the skin type of each portion of the area being examined so as to provide additional information to the dermatologist regarding properties of the skin that may be relevant in choosing and tailoring aesthetic or dermatological treatments.

In various embodiments, the aforementioned processing functions of processor 1330 can be performed in a few seconds or less depending on the type and speed of processor 1330. Thus, real time or near real-time characterization of skin type can be achieved. As later described in more detail, this capability of system 1300 may enable rapid scanning of large areas of skin on the subject before, during, and/or after a dermatological or aesthetic treatment, thereby allowing for the treatment to be uniquely tailored to the individual subject being treated.

Processor 1330, in various embodiments, may additionally or alternatively be configured to control one or more operations of optical sensor 1310 and/or multispectral illumination source 1320. In various embodiments, processor 1330 may be configured to execute instructions stored in memory 1340 for these purposes. For example, processor 1330, in various embodiments, may be configured to instruct optical sensor 1310 in operations such as capturing imagery, adjusting zoom and/or focus of lens 1320, and adjusting modes and filters for capturing imagery in various wavelengths of light. Similarly, processor 1330, in various embodiments, may be configured to instruct multispectral illumination source 1320 in switching between various light sources 1322, 1324, 1326, for example, as well as in adjusting one or more parameters thereof such as brightness.

### Systems and Methods for Characterizing Skin Type Across Multiple Area(s) of the Body

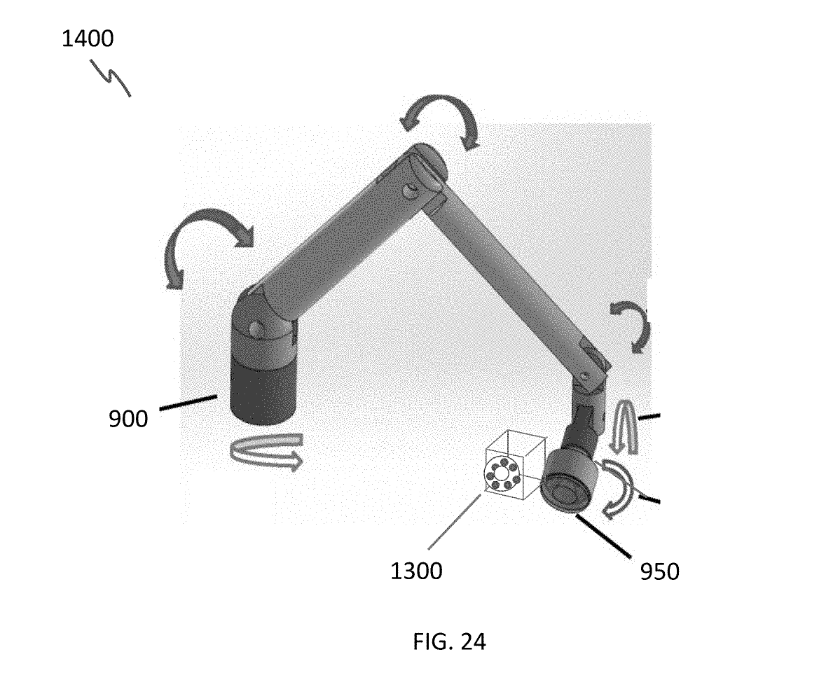

Referring now to FIG. 24, in various embodiments of the present disclosure, system 1300 may be coupled with an articulable arm such as that of apparatus 900, thereby permitting system 1300 to be positioned and oriented for scanning skin on one or more target areas of the patient's body. Such a configuration shall hereinafter be referred to as system 1400.

As shown, in an embodiment of system 1400, system 1300 may be coupled to or integrated with device 950 and oriented in a substantially similar direction as device 950. Such an embodiment could be used for both scanning and treatment. In another embodiment of system 1400 (not shown), system 1300 may replace device 950 at the end of apparatus 900, thereby providing a “scanning only” configuration. In yet another embodiment of system 1400 (not shown), apparatus 900 may be configured to receive either of device 950 and system 1300 depending on whether apparatus 900 is being used for scanning or treatment at a given time.

Positioning apparatus 900, in various embodiments, may be configured to move and/or orient system 1300 to scan all or a portion of a predefined treatment zone, much in the way positioning apparatus may be configured to move device 950 during treatment of a predefined treatment zone. For example, in an embodiment, positioning apparatus 900 may be manually operated to position and orient system 1300 at a distance and angle suitable for measuring features used in determining the skin type of the corresponding area. Positioning apparatus 900 may then be moved to place system 1300 at a second position and orientation for measuring features used in determining the skin type of another area. This may continue in a step-wise or continuous fashion until measurements have been taken throughout the predefined treatment area or desired portion(s) thereof. Likewise, an operator (e.g., clinician or technician) may instead manually move system 1300 to a desired position and orientation, causing apparatus 900 to move in a corresponding manner. As configured, apparatus 900 may primarily act to support system 1300 in the position(s) in which the operator places system 1300, as well as support system 1300 as the operator moves system 1300 to scan various portions of the predefined treatment area. In some embodiments, the computer control provides improved control and movement over stationary or manually operated systems. In particular, computer control may be used to articulate various portions of positioning apparatus 900 so as to direct system 1300 in scanning the predefined target area. Such computer control may be user-directed (e.g., guided by user controls such as a joystick), user-programmed (e.g., user programs a predefined path along which system 1300 is to be directed by the computerized controls), or fully autonomous (e.g., computerized controls determine and implement appropriate movement of apparatus 20, for example, using image recognition technology to identify anatomical features). In an embodiment, as the user directs system 1400 in scanning the target area, system 1400 may record the coordinates of the path and follow it when subsequently implementing the treatment process. As configured, system 1400 will know the skin type of various portions of the target area along the pathway followed during treatment.

Scanning, in various embodiments, may be performed using system 1400 before and/or during treatment. Prior to treatment, in various embodiments, system 1400 may be used to identify skin types across the treatment zone and thereby help a clinician determine appropriate laser and/or cooling settings to use when treating areas with differing skin types. In one such embodiment, system 1400 may be configured to generate a map or other visual aid for depicting information concerning skin type at various locations throughout the target area. For example, color coding could be used to generate a “heat map” type map, using hot shades (e.g., reds, oranges, yellows) to depict locations with more sensitive skin types and cool shades (e.g., violets, blues, greens) to depict locations with less sensitive skin types. Alternatively, a single color could be used and its intensity varied to indicate differences in skin type within the area being examined. In an embodiment, color coding could be presented with some transparency, perhaps with the ability to adjust said transparency, such that a person viewing the map may simultaneously or selectably see the color coding and the skin image itself. In an embodiment, the map may be interactive, allowing the clinician to, for example, select a particular area (e.g., using a mouse or touch screen) to display more detailed information concerning skin type for that particular area. It should be appreciated that such maps or visual aids may be generated from the measurements provided by system 1300 and corresponding location information provided by apparatus 900 using techniques known in the art. Likewise, scanning information collected prior to treatment can be used by system 1400, in some embodiments, to automatically compute appropriate treatment parameters to be used at various locations across the treatment zone according to one or more algorithms, and present these computations to the clinician for facilitating planning efforts. Additionally or alternatively, semi- or fully-autonomous embodiments of system 1400 may utilize skin type information from pre-treatment scans to automatically adapt treatment parameters to the skin type of the particular area being treated throughout the treatment process.

In various embodiments, rather than scanning the entire treatment zone prior to treatment, system 1400 may be configured to scan the various portions of the treatment zone shortly before each is treated. Stated otherwise, system 1400 may be configured to scan a first area then treat the first area, scan a second area then treat the second area, and so on until the entire treatment zone has been treated. Like above, information concerning the skin type of the area about to be treated can be used by the clinician or system 1400 itself to adjust one or more treatment parameters to adapt the treatment to the skin type of that particular area.

It should be recognized that system 1400 may have advantages over traditional devices and methods for assessing skin type. In general, skin typing devices are typically handheld and configured for scanning very small areas of the skin. These limitations can make it burdensome and time consuming (often prohibitively so) to scan enough points in a large target area in order to identify, with suitable fidelity, variations in skin type across the large target area. In contrast, apparatus 900 can direct system 1300 to scan the entirety of a target area(s) while simultaneously taking numerous data samples throughout the process, thereby allowing for precise—and relatively fast—determination of skin type variations across the target area(s).

Further, even to the extent such variations can be identified using manual methods, it can be difficult, if not impossible, to reliably define and relocate the precise locations of such variations and transition zones in between during treatment. In contrast, as apparatus 900 directs system 1300 across the target area, each sample can be automatically associated with the corresponding coordinates of system 1300 (or corresponding coordinates on the skin, as determined using information from sensors 1000 such as orientation angle and distance from the skin) at the time the sample was taken. As configured, variations in skin type can be precisely mapped across the target area, thereby allowing a clinician or system 1300 itself to adjust treatment (e.g., laser power, cooling level, duration of treatment) across the target area to account for corresponding differences in skin type across the area. This ability, in turn, may reduce the amount of time required to treat the area, as well as improve treatment efficacy and safety. It should be appreciated that when treating relatively large areas, these benefits can be very significant and advantageous.

Still further, typical treatments are often performed in a serial manner—that is, fully treat a given area, then move onto the next. While treating a given area, it is often necessary to temporarily turn the treatment laser off or reduce its power to prevent the skin area from overheating. This lengthens the amount of time it takes to treat the given area, and thus the amount of time it takes to treat multiple areas. In contrast, because the present configuration can rapidly determine skin type for multiple areas, apparatus 900 may be configured to treat two or more areas in parallel, even if the areas are characterized by different skin types. In particular, in an embodiment, apparatus 900 may be configured to direct the laser to treat an adjacent area when the temperature of the current area approaches a point at which the laser would traditionally be turned off or decreased in power. As configured, the laser is always treating some portion of the target area, thereby reducing the overall time it takes to treat the target area compared with traditional approaches. Considering that treatment areas often span multiple parts of the body—for example, moving from the abdomen to the hip to the buttocks, thighs, and back—this ability can significantly reduce the overall length of the procedure, potentially treatments that would otherwise require several office visits to require only a single office visit.

System 1400, in various embodiments, may be adapted for measuring and evaluating skin parameters other than skin type to enhance the treatment process. In particular, optical sensor 1310 may be used to capture information concerning the coloration or texture of the skin. In one aspect, during treatment, skin coloration and/or texture could be monitored to provide real-time assessments of the effect the treatment is having on the skin. These real-time assessments may, in turn, be used by the clinician to enhance the efficiency, efficacy, and safety of the treatment. For example, if the area being treated is exhibiting excessive redness or scaling than that expected for a given combination of skin type and treatment parameters, the clinician could adjust laser power, cooling, and/or other treatment parameters to avoid overtreating the area. Conversely, if skin coloration indicates the area being treated is reacting more favorably to the treatment than expected based on skin type, the clinician could adjust treatment parameters (e.g., increase laser power) in an effort to reduce the time required for performing the treatment. In similar fashion, semi-autonomous or autonomous embodiments of system 1400 may evaluate parameters like skin coloration and scaling as captured by optical sensor 1310 and notify a clinician overseeing the treatment. In an embodiment, system 1400 may additionally or alternatively adjust the treatment parameters in response to avoid overtreatment.

While the present disclosure has been described with reference to certain embodiments thereof, it should be understood by those skilled in the art that various changes may be made and equivalents may be substituted without departing from the true spirit and scope of the disclosure. In addition, many modifications may be made to adapt to a particular situation, indication, material and composition of matter, process step or steps, without departing from the spirit and scope of the present disclosure. All such modifications are intended to be within the scope of the claims appended hereto.

What is claimed is:

1. A system for characterizing skin type, the system comprising:
an illumination source configured to generate and direct light of one or more wavelengths onto a skin area;
an optical sensor configured to receive the light reflected from the skin area illuminated by the illumination source and generate a corresponding electronic signal;
a memory containing computer-readable instructions for:
processing the electronic signal to identify one or more properties of the reflected light received by the optical sensor for use in characterizing one or more skin types within the skin area, and
automatically characterizing the one or more skin types within the skin area based at least in part on the one or more identified properties of the reflected light; and
a processor configured to read the computer-readable instructions from the memory and automatically characterize the one or more skin types within the skin area.

2. The system of claim 1, wherein the illumination source includes one or more light emitting diodes (LEDs), laser diodes, incandescent bulbs, and fluorescent lamps, or any combination thereof.

3. The system of claim 1, wherein the one or more wavelengths of light generated by the illumination source includes any one or combination of wavelengths on a spectrum between ultraviolet (UV) and near infrared (NIR), inclusive.

4. The system of claim 1,
wherein the illumination source includes a blackbody radiation source, and
wherein the optical sensor includes one or more spectral filters for selectably filtering one or more wavelengths of reflected light from the skin area illuminated by the blackbody radiation source.

5. The system of claim 1, wherein the optical sensor includes an image sensor, a charged coupled device (CCD) image sensor, a complementary metal-oxide-semiconductor (CMOS) image sensor, a digital camera, or any combination thereof.

6. The system of claim 1, wherein the one or more properties of the reflected light include properties indicative of photo-response by one or more chemical chromophores in the skin area.

7. The system of claim 1, wherein the one or more properties of the reflected light include intensity, color, or a combination thereof.

8. The system of claim 1, wherein skin type includes a characterization of one or more properties of the skin that may contribute to the skin's sensitivity and reaction to one or more wavelengths of light, acids, bases, chemicals, or any combination thereof.

9. The system of claim 1, wherein automatically characterizing the one or more skin types includes evaluating one or more algorithms using, as inputs, measurements of the one or more identified properties.

10. The system of claim 1, wherein automatically characterizing the one or more skin types includes evaluating the one or more properties of the reflected light against the Fitzpatrick Scale.

11. The system of claim 1, wherein the processor automatically characterizes the one or more skin types in the skin area in real-time or near real-time.

12. The system of claim 1, wherein the processor is further configured to:
characterize skin type for multiple portions of the skin area, and
associate the skin type characterization for each of the multiple portions of the skin area with information concerning a location of each of the multiple portions of the skin area.

13. The system of claim 12, wherein the processor is further configured to generate a map or other visual aid for visually presenting variations in the skin type characterizations across the skin area.

14. The system of claim 1,
further including one or more electromagnetic radiation (EMR) sources for generating one or more EMR beams configured for aesthetic or dermatological skin treatment, and
wherein the processor is further configured for a least one of the following:
identify one or more adjustments to one or more parameters of the one or more EMR beams based on the one or more skin type characterizations for presentation to an operator, or
automatically adjust one or more parameters of the one or more EMR beams based on the one or more skin type characterizations.

15. The system of claim 1, further including an articulable arm for positioning at least the optical sensor of the system.

16. The system of claim 15, wherein the processor is further configured to associate the one or more skin type characterizations with at least one of a position and orientation of the optical sensor at the time the optical sensor generated the corresponding electronic signal.

17. A method for characterizing skin type, the method comprising:
illuminating an area of skin with one or more wavelengths of light;
receiving the light reflected from the illuminated skin area and generating a corresponding electronic signal;
processing the electronic signal to identify one or more properties of the reflected light for use in characterizing one or more skin types within the skin area, and
automatically characterizing the one or more skin types within the skin area based at least in part on the one or more identified properties of the reflected light.

18. The method of claim 17, wherein the one or more wavelengths of light includes any one or combination of wavelengths on a spectrum between ultraviolet (UV) and near infrared (NIR), inclusive.

19. The method of claim 17, wherein the one or more properties of the reflected light include properties indicative of photo-response by one or more chemical chromophores in the skin area.

20. The method of claim 17, wherein the one or more properties of the reflected light include intensity, color, or a combination thereof.

21. The method of claim 17, wherein skin type includes a characterization of one or more properties of the skin that may contribute to the skin's sensitivity and reaction to one or more wavelengths of light, acids, bases, chemicals, or any combination thereof.

22. The method of claim 17, wherein automatically characterizing the one or more skin types includes evaluating one or more algorithms using, as inputs, measurements of the one or more identified properties.

23. The method of claim 17, wherein automatically characterizing the one or more skin types includes evaluating the one or more properties of the reflected light against the Fitzpatrick Scale.

24. The method of claim 17, wherein automatically characterizing the one or more skin types in the skin area is performed in real-time or near real-time.

25. The method of claim 17, further including:
characterizing skin type for multiple portions of the skin area, and
associating the skin type characterization for each of the multiple portions of the skin area with information concerning a location of each of the multiple portions of the skin area.

26. The method of claim 25, further including generating a map or other visual aid for visually presenting variations in the skin type characterizations across the skin area.

27. The method of claim 17, further including at least one of:
identifying one or more adjustments to one or more parameters of one or more electromagnetic radiation (EMR) beams used for aesthetic or dermatological treatment of the skin area based on the one or more skin type characterizations for presentation to an operator, and
automatically adjusting one or more parameters of one or more electromagnetic radiation (EMR) beams used for aesthetic or dermatological treatment of the skin area based on the one or more skin type characterizations.

28. The method of claim 17,
wherein an optical sensor receives the light reflected from the illuminated skin area and generates the corresponding electronic signal, and
further including positioning at least the optical sensor of the system using an articulable arm.

29. The method of claim 28, further including associating the one or more skin type characterizations with at least one of a position and orientation of the optical sensor at the time the optical sensor generated the corresponding electronic signal.
